(19) United States
(12) Patent Application Publication
(10) Pub. No.: US 2024/0350465 A1
(43) Pub. Date: Oct. 24, 2024

(54) METHODS FOR SELECTING AN INTRACRANIAL ATHEROSCLEROTIC DISEASE PATIENT FOR TREATMENT

(71) Applicant: UNIVERSITY OF VERMONT AND STATE AGRICULTURAL COLLEGE, Burlington, VT (US)

(72) Inventor: David J. SCHNEIDER, Burlington, VT (US)

(21) Appl. No.: 18/294,996

(22) PCT Filed: Aug. 4, 2021
(86) PCT No.: PCT/US2021/044439

§ 371 Date: Feb. 2, 2024

(51) Int. Cl. A61K 31/4365; A61K 31/405; A61K 31/4433; A61K 31/519; A61K 31/616; A61K 45/06; G01N 15/14; G01N 33/68

(52) U.S. Cl. CPC A61K 31/4365; A61K 31/405; A61K 31/4433; A61K 31/519; A61K 31/616; A61K 45/06; G01N 15/14; G01N 33/6854

(57) ABSTRACT

The present invention features methods for selecting treatment for a patient having suffered a minor stroke or transient ischemic attack (TIA) associated with intracranial atherosclerotic disease (ICAD).

### BACKGROUND OF THE INVENTION

Intracranial atherosclerotic disease (ICAD) can be an important cause of transient ischemic attack (TIA) and minor stroke and can be associated with a high risk of stroke recurrence. Medical therapy can be central to preventing subsequent stroke. A leading medical therapy is dual antiplatelet therapy (DAPT). DAPT (e.g., combining aspirin plus ticagrelor) prevents strokes to a greater extent than using one drug alone; however, the drug combinations increase incidence of severe bleeding. Because medical therapy can entail this tradeoff between the prevention of stroke and the risk of bleeding, clinicians are currently faced with the dilemma of determining their treatment strategy facing these tradeoffs without adequate tools to effectively guide such decisions.

Thus, there remains a need for improved methods for assessing risk and selecting treatments for patients having suffered a stroke and/or a transient ischemic attack (TIA).

### SUMMARY OF THE INVENTION

As described below, the invention features compositions and methods for selecting treatment for a patient having suffered a minor stroke or transient ischemic attack (TIA) associated with intracranial atherosclerotic disease (ICAD). In one aspect, the invention features a method for treating a selected subject. The method involves administering an antithrombotic agent to the selected subject who has previously had at least one stroke, thereby treating the subject. The subject is selected by determining that a level of FcγRIIa protein on platelets from the subject is increased relative to a reference. In embodiments, the subject has intracranial atherosclerotic disease.

In another aspect, the invention features a method for treating a selected subject. The method involves administering an antithrombotic agent to a subject who has intracranial atherosclerotic disease and has had at least one stroke, thereby treating the subject. The subject is selected by determining that a level of FcγRIIa protein on platelets from the subject is increased relative to a reference.

In another aspect, the invention features a method for treating a selected subject who has intracranial atherosclerotic disease and has had at least one stroke. The method involves administering an anti-platelet agent, and/or an anticoagulant to the selected subject. The subject is selected by determining a level of FcγRIIa on platelets from the subject, where a level greater than about 7,500 copies of FcγRIIa per platelet identifies the subject as at risk for subsequent stroke and/or cardiovascular event and in need of antithrombotic therapy.

In another aspect, the invention features a kit for use in the method of any one of the above aspects, where the kit contains a FcγRIIa protein capture reagent.

The method of claim 43, further comprising quantifying the number of molecules of FcγRIIa protein on individual platelets.

In any of the above aspects, the method further involves quantifying the number of molecules of FcγRIIa on individual platelets.

In any of the above aspects, the stroke is a minor stroke and/or a transient ischemic attack.

In any of the above aspects, the antithrombotic agent is selected from one or more of a small molecule compound, an inhibitory nucleic acid, and an antibody or antigen-binding fragment thereof. In embodiments, the inhibitory nucleic acid is selected from one or more of an antisense molecule, an shRNA, and an siRNA.

In any of the above aspects, the antithrombotic agent contains an antiplatelet agent or an anticoagulant. In any of the above aspects, the method involves administering at least two antithrombotic agents to the subject.

In any of the above aspects, the agent contains an adenosine diphosphate (ADP) receptor antagonist and/or a protease-activated receptor (PAR) antagonist.

In any of the above aspects, the anti-thrombotic agent contains an ADP receptor antagonist. In embodiments, the ADP receptor antagonist targets P2Y₁₂. In embodiments, the ADP receptor antagonist contains a small molecule compound. In embodiments, the ADP receptor antagonist contains a thienopyridine. In embodiments, the thienopyridine contains prasugrel, clopidogrel, ticagrelor, or ticlopidine.

In any of the above aspects, the antithrombotic agent contains a PAR antagonist. In embodiments, the PAR antagonist targets PARI, PAR3, or PAR4. In embodiments, the PAR antagonist targets PARI. In embodiments, the PAR antagonist contains a small molecule compound. In embodiments, the PAR antagonist contains vorapaxar. In embodiments, the antithrombotic agent contains acetylsalicylic acid (ASA), dipyridamole, and/or eptifibatide.

In any of the above aspects, the antithrombotic agent contains an anticoagulant agent. In embodiments, the anticoagulant is an inhibitor of factor XIa. In embodiments, the anticoagulant contains apixaban, argatroban, betrixaban, bivalirudin, dabigatran, desirudin, edoxaban, enoxaparin, heparin, reteplase, rivaroxaban, and/or warfarin.

In any of the above aspects, the level of FcyRIIa protein on platelets is determined using an assay selected from one or more of flow cytometry, immunoassay, ELISA, western blotting, and radioimmunoassay. In any of the above aspects, the level of FcγRIIa protein on the platelets is determined using flow cytometry. In any of the above aspects, the level of FcyRIIa protein on platelets is determined using fluorometric or colorimetric assay. In any of the above aspects, determining the level of FcγRIIa protein on the platelets involves contacting the platelets with a capture reagent. In embodiments, the capture reagent contains an anti-FcγRIIa protein antibody or antigen-binding fragment thereof containing a detectable label. In embodiments, the detectable label contains a fluorochrome. In embodiments, the capture reagent contains a fluorochrome-labeled antibody.

In any of the above aspects, the reference is a healthy subject that has not had a stroke. In any of the above aspects, the reference is a healthy subject that does not have intracranial atherosclerotic disease.

In any of the above aspects, the increase is by at least about 1.5, 2, 3, 4, or 5-fold. In any of the above aspects, the level of FcγRIIa protein on platelets is increased relative to the reference if greater than about 7,500, 8,000, 9,000, or 10,000 FcyRIIa protein molecules per platelet. In any of the above aspects, the level of FcγRIIa protein on platelets is increased relative to the reference if greater than about 8,000 FcyRIIa protein molecules per platelet. In any of the above aspects, the level of FcγRIIa protein on platelets is increased relative to the reference if greater than about 11,000 FcyRIIa protein molecules per platelet.

In any of the above aspects, the subject is selected only if the level of FcγRIIa on platelets from the subject is determined to be equal to or greater than about 11,000 copies of FcγRIIa per platelet at two time points. In embodiments, the time points are separated by at least about one day. In embodiments, the time points are separated by at least about 7 days.

In any of the above aspects, incidence and/or severity of a cardiovascular event is reduced. In any of the above aspects, incidence and/or severity of a subsequent stroke is reduced. In any of the above aspects, incidence of death is reduced.

In any of the above aspects, the anti-platelet agent contains an Adenosine diphosphate (ADP) receptor antagonist, or a Protease-activated receptor (PAR) antagonist. In any of the above aspects, the Adenosine diphosphate (ADP) receptor antagonist and/or the Protease-activated receptor (PAR) antagonist contains one or more of prasugrel, ticagrelor, clopidogrel, and vorapaxar. In any of the above aspects, the anti-platelet agent contains acetylsalicylic acid (ASA), dipyridamole, or eptifibatide. In any of the above aspects, the anticoagulant contains one or more of apixaban, argatroban, betrixaban, bivalirudin, dabigatran, desirudin, edoxaban, enoxaparin, heparin, reteplase, rivaroxaban, and warfarin.

In any of the above aspects, the level of the FcγRIIa is determined by contacting a sample containing platelets from the subject with an FcγRIIa-binding conjugate to form a bound complex of the FcγRIIa-binding conjugate and an FcγRIIa protein molecule on the surface of the platelets, and detecting binding between the FcγRIIa-binding conjugate and the FcγRIIa protein molecule. In embodiments, the FcγRIIa-binding conjugate is an anti-FcγRIIa antibody.

The invention provides compositions and methods for selecting treatment for a patient having suffered a minor stroke or transient ischemic attack (TIA) associated with intracranial atherosclerotic disease (ICAD). Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “FcγRIIa protein” is meant a low affinity immunoglobulin gamma Fc region receptor II-a having at least about 85% identity to GenBank Accession No. NP_001129691.1 or a fragment thereof that binds an IgG protein. An exemplary amino acid sequence of the FcγRIIa is provided at GenBank Accession No. NP_001129691.1:

MTMETQMSQNVCPRNLWLLQPLTVLLLLASADSQAAAPPKAVLKLEPPW
INVLQEDSVTLTCQGARSPESDSIQWFHNGNLIPTHTQPSYRFKANNND
SGEYTCQTGQTSLSDPVHLTVLSEWLVLQTPHLEFQEGETIMLRCHSWK
DKPLVKVTFFQNGKSQKFSHLDPTFSIPQANHSHSGDYHCTGNIGYTLF
SSKPVTITVQVPSMGSSSPMGIIVAVVIATAVAAIVAAVVALIYCRKKR
ISANSTDPVKAAQFEPPGRQMIAIRKRQLEETNNDYETADGGYMTLNPR
APTDDDKNIYLTLPPNDHVNSNN.

By “FcγRIIa polynucleotide” is meant a nucleic acid molecule encoding an FcγRIIa protein. An exemplary nucleic acid molecule encoding an FcγRIIa protein is provided at GenBank Accession No. NM_001136219.1:

CTCTTTTCTAAGCTTGTCTCTTAAAACCCACTGGACGTTGGCACAGTGC
TGGGATGACTATGGAGACCCAAATGTCTCAGAATGTATGTCCCAGAAAC
CTGTGGCTGCTTCAACCATTGACAGTTTTGCTGCTGCTGGCTTCTGCAG
ACAGTCAAGCTGCAGCTCCCCCAAAGGCTGTGCTGAAACTTGAGCCCCC
GTGGATCAACGTGCTCCAGGAGGACTCTGTGACTCTGACATGCCAGGGG
GCTCGCAGCCCTGAGAGCGACTCCATTCAGTGGTTCCACAATGGGAATC
TCATTCCCACCCACACGCAGCCCAGCTACAGGTTCAAGGCCAACAACAA
TGACAGCGGGGAGTACACGTGCCAGACTGGCCAGACCAGCCTCAGCGAC
CCTGTGCATCTGACTGTGCTTTCCGAATGGCTGGTGCTCCAGACCCCTC
ACCTGGAGTTCCAGGAGGGAGAAACCATCATGCTGAGGTGCCACAGCTG
GAAGGACAAGCCTCTGGTCAAGGTCACATTCTTCCAGAATGGAAAATCC
CAGAAATTCTCCCATTTGGATCCCACCTTCTCCATCCCACAAGCAAACC
ACAGTCACAGTGGTGATTACCACTGCACAGGAAACATAGGCTACACGCT
GTTCTCATCCAAGCCTGTGACCATCACTGTCCAAGTGCCCAGCATGGGC
AGCTCTTCACCAATGGGGATCATTGTGGCTGTGGTCATTGCGACTGCTG
TAGCAGCCATTGTTGCTGCTGTAGTGGCCTTGATCTACTGCAGGAAAAA
GCGGATTTCAGCCAATTCCACTGATCCTGTGAAGGCTGCCCAATTTGAG
CCACCTGGACGTCAAATGATTGCCATCAGAAAGAGACAACTTGAAGAAA
CCAACAATGACTATGAAACAGCTGACGGCGGCTACATGACTCTGAACCC
CAGGGCACCTACTGACGATGATAAAAACATCTACCTGACTCTTCCTCCC
AACGACCATGTCAACAGTAATAACTAAAGAGTAACGTTATGCCATGTGG
TCATACTCTCAGCTTGCTGAGTGGATGACAAAAAGAGGGGAATTGTTAA
AGGAAAATTTAAATGGAGACTGGAAAAATCCTGAGCAAACAAAACCACC
TGGCCCTTAGAAATAGCTTTAACTTTGCTTAAACTACAAACACAAGCAA
AACTTCACGGGGTCATACTACATACAAGCATAAGCAAAACTTAACTTGG
ATCATTTCTGGTAAATGCTTATGTTAGAAATAAGACAACCCCAGCCAAT
CACAAGCAGCCTACTAACATATAATTAGGTGACTAGGGACTTTCTAAGA
AGATACCTACCCCCAAAAAACAATTATGTAATTGAAAACCAACCGATTG
CCTTTATTTTGCTTCCACATTTTCCCAATAAATACTTGCCTGTGACATT
TTGCCACTGGAACACTAAACTTCATGAATTGCGCCTCAGATTTTTCCTT
TAACATCTTTTTTTTTTTTGACAGAGTCTCAATCTGTTACCCAGGCTGG
AGTGCAGTGGTGCTATCTTGGCTCACTGCAAACCCGCCTCCCAGGTTTA
AGCGATTCTCATGCCTCAGCCTCCCAGTAGCTGGGATTAGAGGCATGTG
CCATCATACCCAGCTAATTTTTGTATTTTTTATTTTTTTTTTTTAGTAG
AGACAGGGTTTCGCAATGTTGGCCAGGCCGATCTCGAACTTCTGGCCTC
TAGCGATCTGCCCGCCTCGGCCTCCCAAAGTGCTGGGATGACCAGCATC
AGCCCCAATGTCCAGCCTCTTTAACATCTTCTTTCCTATGCCCTCTCTG
TGGATCCCTACTGCTGGTTTCTGCCTTCTCCATGCTGAGAACAAAATCA
CCTATTCACTGCTTATGCAGTCGGAAGCTCCAGAAGAACAAAGAGCCCA
GACTCTTCCAGAGTCATCTACCTGAGTCCCAAAGCTCCCTGTCCTGAAA
GATTACCAGAACCACATTAAGTCTCCATTGTTTTGCCTTGGGATTTGAG
AAGAGAATTAGAGAGGTGAGGATCTGGTATTTCCTGGACTAAATTCCCC
TTGGGGAAGACGAAGGGATGCTGCAGTTCCAAAAGAGAAGCCACAGACA
ATATGGTCCCAAATGACTGACTGCACCTTCTGTGCCTCAGCCGTTCTTG
ACATCAAGAATCTTCTGTTCCACATCCACACAGCCAATACAATTAGTCA
AACCACTGTTATTAACAGATGTAGCAACATGAGAAACGCTTATGTTACA
GGTTACATGAGAGCAATCATGTAAGTCTATATGACTTCAGAAATGTTAA
AATAGACTAACCTCTAACAACAAATTAAAAGTGATTGTTTCAAGGTGAT
GCAATTATTGATGACCTATTTTATTTTTCTATAATGATCATATATTACC
TTTGTAATAAAACATTATAACCAAAACA.

By “FcγRIIa protein specific agent” is meant any small molecule compound, antibody, nucleic acid molecule, or protein, or fragments thereof that specifically bind to a FcγRIIa protein. A non-limiting example of an FcγRIIa protein specific agent is an anti-FcγRIIa protein antibody, or a fragment thereof.

By “Protease-activated receptor (PAR) protein” is meant a G protein-coupled receptor that is activated by cleavage of a portion of its extracellular domain. PARs are present at high levels in platelets. PARs include the thrombin receptors PARI, PAR3 and PAR4. PARs are activated by the action of serine proteases such as thrombin (e.g., activating PARs 1, 3 and 4). Cleavage of the N-terminus of the receptor, generates a tethered ligand (SFLLRN) that acts as an agonist, causing a physiological response. The cellular effects of thrombin are mediated by protease-activated receptors (PARs). Thrombin signaling in platelets contributes to hemostasis and thrombosis. Thrombin receptor antagonists include vorapaxar (SCH 530348) which is a PARI antagonist.

By “Adenosine diphosphate (ADP) receptor protein” is meant a purinergic G protein-coupled receptors, stimulated by the nucleotide Adenosine diphosphate (ADP). ADP receptors include P2Y₁₂ which regulates thrombosis. Adenosine diphosphate (ADP) receptor antagonists are agents that inhibit adenosine diphosphate receptors. P2Y₁₂ is the target of the anti-platelet drugs including prasugrel, clopidogrel, ticagrelor, ticlopidine, and other thienopyridines.

By “acetylsalicylic acid (ASA)” is meant a compound corresponding to CAS No. 50-78-2 and having the structure

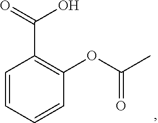

or a pharmaceutically acceptable salt or solvate thereof. Acetylsalicylic acid (ASA) is a platelet aggregation inhibitor.

By “clopidogrel” is meant a compound corresponding to CAS No. 113665-84-2 and having the structure

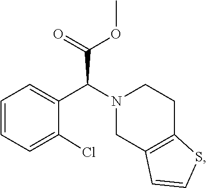 [SMILES: COC(=O)[C@H](c1ccccc1Cl)N1CCc2sccc2C1]

or a pharmaceutically acceptable salt or solvate thereof. Clopidogrel is a potent platelet aggregation inhibitor.

By “dipyridamole” is meant a compound corresponding to CAS No. 58-32-2 and having the structure

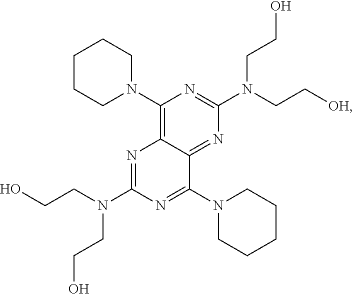 [SMILES: OCCN(CCO)c1nc(N2CCCCC2)c2nc(N(CCO)CCO)nc(N3CCCCC3)c2n1]

or a pharmaceutically acceptable salt or solvate thereof. Dipyridamole is a platelet aggregation inhibitor.

By “eptifibatide” is meant a compound corresponding to CAS No. 188627-80-7 and having the structure

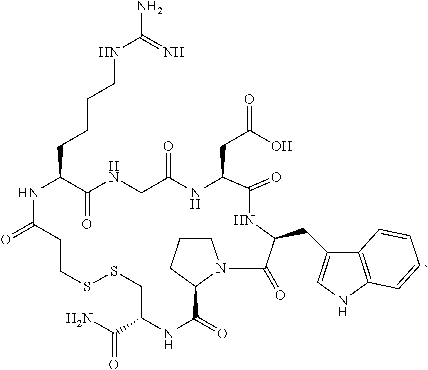 [SMILES: N=C(N)NCCCC[C@@H]1NC(=O)CCSSC[C@@H](C(N)=O)NC(=O)[C@H]2CCCN2C(=O)[C@H](Cc2c[nH]c3ccccc23)NC(=O)[C@H](CC(=O)O)NC(=O)CNC1=O]

or a pharmaceutically acceptable salt or solvate thereof. Eptifibatide is a platelet aggregation inhibitor.

By “prasugrel” is meant a compound corresponding to CAS No. 150322-43-3 and having the structure

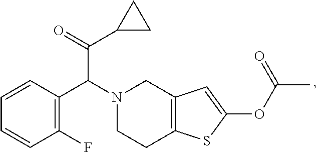 [SMILES: CC(=O)Oc1cc2c(s1)CCN(C(C(=O)C1CC1)c1ccccc1F)C2]

or a pharmaceutically acceptable salt or solvate thereof. Prasugrel is a potent platelet aggregation inhibitor.

By “ticagrelor” is meant a compound corresponding to CAS No. 274693-27-5 and having the structure

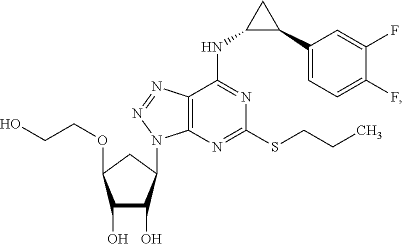

or a pharmaceutically acceptable salt or solvate thereof. Ticagrelor is a potent platelet aggregation inhibitor.

By “ticlopidine” is meant a compound corresponding to CAS No. 55142-85-3 and having the structure

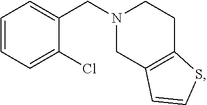 [SMILES: Clc1ccccc1CN1CCc2sccc2C1]

or a pharmaceutically acceptable salt or solvate thereof. Ticlopidine is a potent platelet aggregation inhibitor.

By “vorapaxar” is meant a compound corresponding to CAS No. 618385-01-6 and having the structure

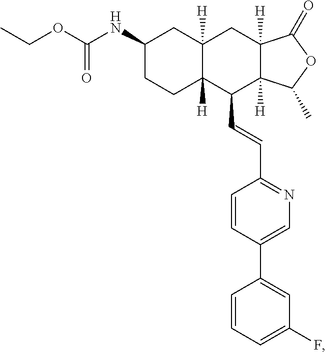 [SMILES: CCOC(=O)N[C@@H]1CC[C@@H]2[C@@H](C1)C[C@H]1C(=O)O[C@H](C)[C@H]1[C@H]2/C=C/c1ccc(-c2cccc(F)c2)cn1]

or a pharmaceutically acceptable salt or solvate thereof. Vorapaxar is a potent platelet aggregation inhibitor.

By “apixaban” is meant a compound corresponding to CAS No. 503612-47-3 and having the structure

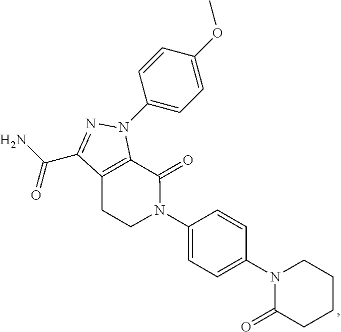 [SMILES: COc1ccc(-n2nc(C(N)=O)c3c2C(=O)N(c2ccc(N4CCCCC4=O)cc2)CC3)cc1]

or a pharmaceutically acceptable salt or solvate thereof. Apixaban is an anticoagulant.

By “argatroban” is meant a compound corresponding to CAS No. 74863-84-6 and having the structure

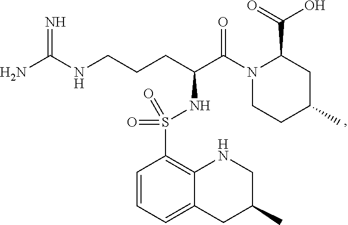 [SMILES: C[C@@H]1CNc2c(cccc2S(=O)(=O)N[C@@H](CCCNC(=N)N)C(=O)N2CC[C@@H](C)C[C@@H]2C(=O)O)C1]

or a pharmaceutically acceptable salt or solvate thereof. Argatroban is an anticoagulant.

By “betrixaban” is meant a compound corresponding to CAS No. 330942-05-7 and having the structure

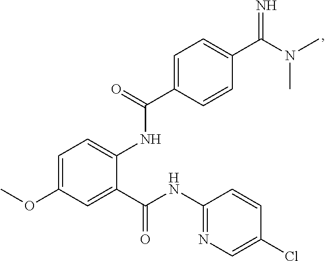

or a pharmaceutically acceptable salt or solvate thereof. Betrixaban is an anticoagulant.

By “bivalirudin” is meant a compound corresponding to CAS No. 128270-60-0 and having the structure

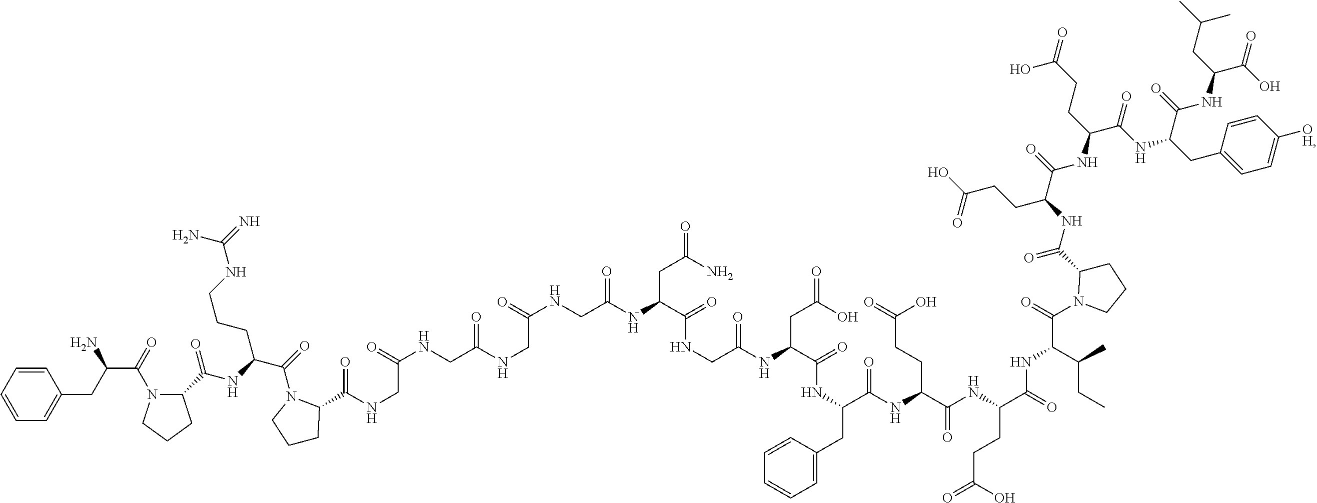 [SMILES: CC[C@H](C)[C@H](NC(=O)[C@H](CCC(=O)O)NC(=O)[C@H](CCC(=O)O)NC(=O)[C@H](Cc1ccccc1)NC(=O)[C@H](CC(=O)O)NC(=O)CNC(=O)[C@H](CC(N)=O)NC(=O)CNC(=O)CNC(=O)CNC(=O)CNC(=O)[C@@H]1CCCN1C(=O)[C@H](CCCNC(=N)N)NC(=O)[C@@H]1CCCN1C(=O)[C@H](N)Cc1ccccc1)C(=O)N1CCC[C@H]1C(=O)N[C@@H](CCC(=O)O)C(=O)N[C@@H](CCC(=O)O)C(=O)N[C@@H](Cc1ccc(O)cc1)C(=O)N[C@@H](CC(C)C)C(=O)O]

or a pharmaceutically acceptable salt or solvate thereof. Bivalirudin is an anticoagulant.

By “dabigatran” is meant a compound corresponding to CAS No. 211915-06-9 and having the structure

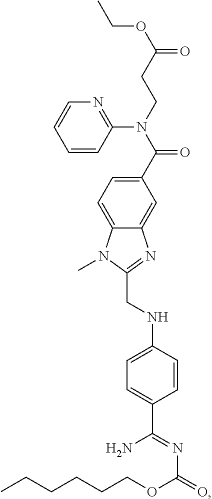 [SMILES: CCCCCCOC(=O)/N=C(\N)c1ccc(NCc2nc3cc(C(=O)N(CCC(=O)OCC)c4ccccn4)ccc3n2C)cc1]

or a pharmaceutically acceptable salt or solvate thereof. Dabigatran is an anticoagulant.

By “desirudin” is meant a peptide having at least 85% sequence identity to the amino acid sequence VVYTDCTESGQNLCLCEGSNVCGQGNKCILGSDGEKNQCVTGEGTPKPQSHNDGDFEEI PEEYLQ (SEQ ID NO: 1). In embodiments, the amino acid sequence of desirudin has at least about 90%, 95%, or 99% sequence identity to SEQ ID NO: 1. In embodiments, the amino acid sequence of desirudin contains 1, 2, 3, 4, 5, 6, 7, 8, 9, or 10 alterations relative to SEQ ID NO: 1. Desirudin is an anticoagulant.

By “edoxaban” is meant a compound corresponding to CAS No. 480449-70-5 and having the structure

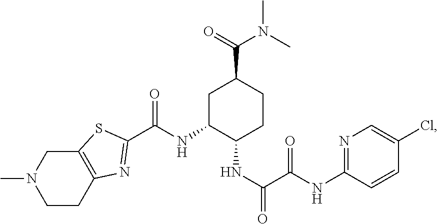 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

or a pharmaceutically acceptable salt or solvate thereof. Edoxaban is an anticoagulant.

By “enoxaparin” is meant a compound corresponding to CAS No. 679809-58-6 and having the structure

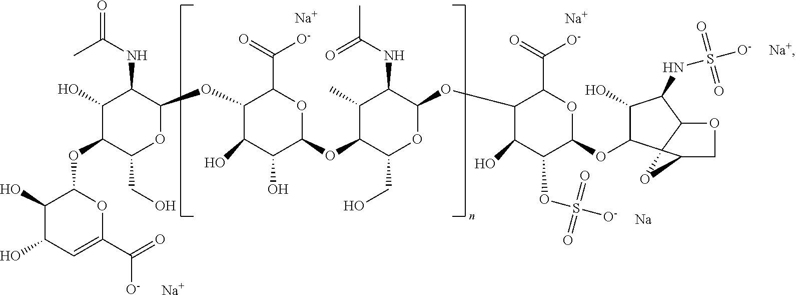

or a pharmaceutically acceptable salt or solvate thereof. Enoxaparin is an anticoagulant.

By “heparin” is meant a compound corresponding to CAS No. 9005-49-6 and having the structure

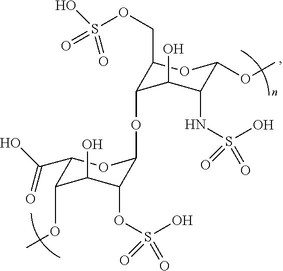

or a pharmaceutically acceptable salt or solvate thereof. Heparin is an anticoagulant.

By “reteplase” is meant a peptide having at least 85% sequence identity to the amino acid sequence SYQGNSDCYFGNGSAYRGTHSLTESGASCLPWNSMILIGKVYTAQNPSAQALGLGKHN YCRNPDGDAKPWCHVLKNRRLTWEYCDVPSCSTCGLRQYSQPQFRIKGGLFADIASHP WQAAIFAKHRRSPGERFLCGGILISSCWIL SAAHCFQERFPPHHLTVILGRTYRVVPGEEE QKFEVEKYIVHKEFDDDTYDNDIALLQLKSDSSRCAQESSVVRTVCLPPADLQLPDWTE CELSGYGKHEALSPFYSERLKEAHVRLYPSSRCTSQHLLNRTVTDNMLCAGDTRSGGPQ ANLHDACQGDSGGPLVCLNDGRMTLVGIISWGLGCGQKDVPGVYTKVTNYLDWIRDN MRP (SEQ ID NO: 2). In embodiments, the amino acid sequence of reteplase has at least about 90%, 95%, or 99% sequence identity to SEQ ID NO: 2. In embodiments, the amino acid sequence of reteplase contains 1, 2, 3, 4, 5, 6, 7, 8, 9, or 10 alterations relative to SEQ ID NO: 2. Reteplase is an anticoagulant.

By “rivaroxaban” or is meant a compound corresponding to CAS No. 366789-02-8 and having the structure

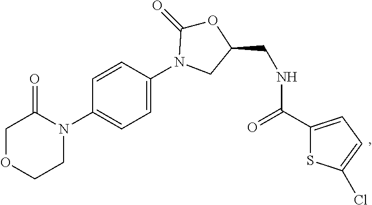 [SMILES: O=C(NC[C@H]1CN(c2ccc(N3CCOCC3=O)cc2)C(=O)O1)c1ccc(Cl)s1]

or a pharmaceutically acceptable salt or solvate thereof. Rivaroxaban is an anticoagulant.

By “warfarin” is meant a compound corresponding to CAS No. 81-81-2 and having the structure

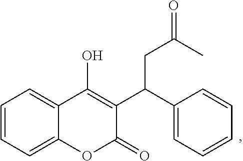 [SMILES: CC(=O)CC(c1ccccc1)c1c(O)c2ccccc2oc1=O]

or a pharmaceutically acceptable salt or solvate thereof. Warfarin is an anticoagulant. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977): 1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

By “antithrombotic agent” is meant an agent suitable for use in an antithrombotic therapy. Non-limiting examples of antithrombotic agents include anti-platelet drugs, such as PAR antagonists (e.g., vorapaxar), ADP receptor antagonists (e.g., clopidogrel, prasugrel, ticagrelor, ticlopidine, and other thienopyridines), acetylsalicylic acid (ASA), dipyridamole, and eptifibatide. Further non-limiting examples of antithrombotic agents include anticoagulants, such as apixaban, argatroban, betrixaban, bivalirudin, dabigatran, desirudin, edoxaban, enoxaparin, heparin, reteplase, rivaroxaban, warfarin, and factor XIa (FXIa) inhibitors (e.g., those described in Rami A. Al-Horani and Umesh R. Desani, Expert Opin Ther Pat. 26:323-345 (2016)).

By “antithrombotic therapy” is meant any treatment used to inhibit or reduce thrombosis in a subject. In embodiments, antithrombotic therapy involves administering an anti-platelet agent and/or an anticoagulant to a subject.

By “agent” is meant any small molecule chemical compound, antibody, nucleic acid molecule, or protein, or fragments thereof. In embodiments, the agent is an antithrombotic agent.

By “ameliorate” is meant decrease, suppress, attenuate, diminish, arrest, or stabilize the development or progression of a disease.

By “alteration” is meant a change (increase or decrease) in the sequence, level, or activity of a polynucleotide or protein as detected by standard art known methods such as those described herein. As used herein, an alteration includes a 10% change in levels, preferably a 25% change, more preferably a 40% change, and most preferably a 50% or greater change in levels.”

By “analog” is meant a molecule that is not identical, but has analogous functional or structural features to a reference molecule. For example, a protein analog retains the biological activity of a corresponding naturally-occurring protein, while having certain biochemical modifications that enhance the analog's function relative to a naturally occurring protein. Such biochemical modifications could increase the analog's protease resistance, membrane permeability, or half-life, without altering, for example, ligand binding. An analog may include an unnatural amino acid.

By “capture reagent” is meant a reagent that specifically binds a protein or nucleic acid molecule. In various embodiments, the capture reagent for an FcγRIIa protein is an anti-FcγRIIa protein antibody.

By “cardiovascular event” is meant an incident that can be associated with damage to the heart muscle. Non-limiting examples of cardiovascular events include a myocardial infarction (MI; i.e., heart attack) and heart failure.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected. In embodiments, the analyte is an FcγRIIa protein. By “analyte-binding conjugate” is meant a detectable molecule that binds a compound to be detected. In embodiments, an analyte-binding conjugate is an anti-FcγRIIa antibody comprising a detectable label.

By “detectable label” is meant a composition that when linked to a molecule of interest renders the latter detectable. In some embodiments, the detectable label is detected via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Non-limiting examples of diseases include a transient ischemic attack (TIA), intracranial atherosclerotic disease (ICAD), and stroke.

By “effective amount” is meant the amount of an agent effective in ameliorating the symptoms and/or incidence of a disease relative to an untreated patient. In some embodiments, an effective amount of an agent is an amount useful to stabilize, reduce, or eliminate a thrombus or the propensity to develop a thrombus. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. Ameliorating the incidence of a disease can include decreasing or eliminating frequency of incidence of an event (e.g., stroke, cardiovascular event, or death).

By “fragment” is meant a portion of a protein or nucleic acid molecule. This portion contains, preferably, at least 10%, 20%, 30%, 40%, 50%, 60%, 70%, 80%, or 90% of the entire length of the reference nucleic acid molecule or protein. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100% relative to a reference.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional protein sequence.

By an “isolated protein” is meant a protein of the invention that has been separated from components that naturally accompany it. Typically, the protein is isolated when it is at least 60%, by weight, free from the proteins and naturally-occurring organic molecules with which it is naturally associated. Preferably, the preparation is at least 75%, more preferably at least 90%, and most preferably at least 99%, by weight, a protein of the invention. An isolated protein of the invention may be obtained, for example, by extraction from a natural source, by expression of a recombinant nucleic acid encoding such a protein; or by chemically synthesizing the protein. Purity can be measured by any appropriate method, for example, column chromatography, polyacrylamide gel electrophoresis, or by HPLC analysis.

By “marker” is meant any protein, polynucleotide, or clinical parameter comprising an alteration that is associated with a disease or disorder. In some embodiments, the alteration is a change in level, activity or secondary modification. For example, an increase in FcγRIIa protein level, activity, phosphorylation, and/or expression is likely associated with increased platelet reactivity, and/or increased incidence of stroke, cardiovascular event, and/or death.

As used herein, “obtaining” as in “obtaining an agent” includes synthesizing, purchasing, or otherwise acquiring the agent.

By “protein” or “amino acid sequence” is meant any chain of amino acids, regardless of length or post-translational modification. In embodiments, the protein comprises post-translational modifications. In embodiments, a protein is a single molecule. In various embodiments, the post-translational modification is glycosylation or phosphorylation. In various embodiments, conservative amino acid substitutions may be made to a protein to provide functionally equivalent variants, or homologs of the protein. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering protein sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and proteins disclosed herein.

By “reduces” is meant a negative alteration of at least 10%, 25%, 50%, 75%, or 100%.

By “reference” is meant a standard or control condition. A non-limiting example of a reference is a healthy subject or a sample collected from a healthy subject. In embodiments, the healthy subject has not suffered a stroke, cardiovascular event, or death and/or the subject has not suffered a subsequent stroke, cardiovascular event, or death following a previous stroke or cardiovascular event.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For proteins, the length of the reference protein sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These double stranded RNAs (dsRNAs) can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a protein of the invention, but which does not substantially recognize and bind other molecules in a sample, for example, a biological sample, which naturally includes a protein of the invention.

By “stroke” is meant an interruption of blood flow to the central nervous system resulting in neuronal cell death. Vascular causes of stroke include, but are not limited to cerebral infarction, intracerebral hemorrhage (ICH), and subarachnoid hemorrhage (SAH). See also Sacco et al., An updated definition of stroke for the 21st century, Stroke 2013; 44:2064-89 and Table 1 below for non-limiting examples of forms of stroke. In embodiments, the stroke is a minor stroke. In embodiments, a subject having a minor stroke has a score of 1 on every National Institutes Health Stroke Scale (NIHSS) item and normal consciousness and/or an NIHSS≤1 3 (see, Fischer, et al, “What Is a Minor Stroke?”, Stroke, 41:661-666 (2010). In embodiments, a subject having a minor stroke has an NIHSS of from 0 to 3 and one or more of deficit not measurable on the NIHSS, pure sensory stroke, isolated ataxia, isolated dysarthria, or isolated facial weakness (see Strambo, et al., “Defining minor symptoms in acute ischemic stroke”, Cerebrovascular Diseases, 39-209-215 (2015)). In embodiments, a subject having a minor stroke has an NIHSS of from 0 to 1 and one or more of a deficit not measurable on the NIHSS or a deficit resulting in total N CISS score=1 with the point assigned in the level of consciousness (LOC), gaze, facial palsy, sensory, or dysarthria (see Strambo, et al.). In embodiments, a subject having a minor stroke has an NIHSS of ≤3. In embodiments, a subject having a minor stroke has an NIHSS of ≤6 with preservation of level of consciousness (LOC) items, score≤1 in cortical (language and visual field) and motor items (limbs and speech), score 0 in the motor item of dominant arm, and any score in the remaining items (see Strambo, et al.).

TABLE 1
Forms of Stroke

Central nervous system (CNS) infarction: CNS infarction is brain, spinal
cord, or retinal cell death attributable to ischemia, based on
1. pathological, imaging, or other objective evidence of cerebral, spinal cord,
or retinal focal ischemic injury in a defined vascular distribution; or
2. clinical evidence of cerebral, spinal cord, or retinal focal ischemic injury
based on symptoms persisting ≥24 hours or until death, and other
etiologies excluded. (Note: CNS infarction includes hemorrhagic
infarctions, types I and II)
Ischemic stroke: An episode of neurological dysfunction caused by focal
cerebral, spinal, or retinal infarction. (Note: Evidence of CNS infarction is
defined above.)
Silent CNS infarction: Imaging or neuropathological evidence of CNS
infarction, without a history of acute neurological dysfunction attributable to
the lesion.
Intracerebral hemorrhage: A focal collection of blood within the brain
parenchyma or ventricular system that is not caused by trauma.
(Note: Intracerebral hemorrhage includes parenchymal hemorrhages after CNS
infarction, types I and II)
Stroke caused by intracerebral hemorrhage: Rapidly developing clinical
signs of neurological dysfunction attributable to a focal collection of blood
within the brain parenchyma or ventricular system that is not caused by
trauma.
Silent cerebral hemorrhage: A focal collection of chronic blood products
within the brain parenchyma, subarachnoid space, or ventricular system on
neuroimaging or neuropathological examination that is not caused by trauma
and without a history of acute neurological dysfunction attributable to the
lesion.
Subarachnoid hemorrhage: Bleeding into the subarachnoid space (the space
between the arachnoid membrane and the pia mater of the brain or spinal
cord).
Stroke caused by subarachnoid hemorrhage: Rapidly developing signs of
neurological dysfunction and/or headache because of bleeding into the
subarachnoid space (the space between the arachnoid membrane and the pia
mater of the brain or spinal cord), which is not caused by trauma.
Stroke caused by cerebral venous thrombosis: Infarction or hemorrhage in
the brain, spinal cord, or retina because of thrombosis of a cerebral venous
structure. Symptoms or signs caused by reversible edema without infarction or
hemorrhage do not qualify as stroke.
Stroke not otherwise specified: An episode of acute neurological dysfunction
presumed to be caused by ischemia or hemorrhage, persisting ≥24 hours or until
death, but without sufficient evidence to be classified as one of the above.

By “transient ischemic attack (TIA)” is meant an interruption in blood supply to the central nervous system resulting in transient neurological dysfunction. In embodiments, a TIA is associated with reversible neurological dysfunction.

Nucleic acid molecules useful in the methods of the invention include any nucleic acid molecule that encodes a protein of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a protein of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. A treatment can involve amelioration of at least one symptom of and/or reduce risk, occurrence, subsequent occurrence, or recurrence of stroke, cardiovascular event, and/or death. A non-limiting example of a cardiovascular event is a heart attack.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a bar graph showing activation of platelets identified by surface levels of P-selectin with the use of flow cytometry. Patients with end stage renal disease (n=33) were stratified into 2 groups on the basis of median levels of FcγRIIa proteins. The percentage of platelets having P-selectin on their surface in the absence of agonist and in response to the collagen mimetic convulxin (1 ng/ml), platelet activating factor (PAF, 1 nM), adenosine diphosphate (ADP 0.2 μM), or thrombin (1 nM) is shown. High platelet levels of FcγRIIa protein was associated with greater activation of platelets in response to each agonist.

FIG. 2 provides a schematic summarizing study design.

FIG. 3 provides Kaplan-Meier curves of the probability of freedom from heart attack, stroke and death. The average duration of follow-up was 20 months (range 6-29 months).

FIG. 4 provides a schematic illustration of FcγRIIa protein platelet activation. Not being bound by theory, clustering of FcγRIIa proteins leads to phosphorylation, which leads to downstream signaling and activation of platelets. FcγRIIa proteins can amplify activation of platelets in response to a stimulus or agonist. Not being bound by theory, because platelet FcγRIIa protein level reflects megakaryocyte production, FcγRIIa protein level does not exhibit the magnitude of variation that is seen with platelet function. Thus, FcγRIIa proteins can be an ideal biomarker of consistently high or elevated platelet activity.

### DETAILED DESCRIPTION OF THE INVENTION

The invention features compositions and methods that are useful for assessing risk for stroke in a subject, and for treatment selection. In embodiments, the subject has suffered a stroke, transient ischemic attack (TIA).

The invention is based, at least in part, on the discovery that FcγRIIa proteins are a novel biomarker, the quantification of which allows for identification of patients at high and low risk of stroke and cardiovascular events. The stroke and cardiovascular events can be subsequent events following a previous event. Not being bound by theory, FcγRIIa protein is a marker of increased platelet reactivity. Greater platelet reactivity can be associated with a greater risk of cardiovascular events or stroke. The methods provided herein can reproducibly identify increased platelet reactivity. The methods provided herein involve using levels of FcγRIIa proteins on the surface of platelets to identify patients at high and low residual cardiovascular and/or stroke risk and effectively guide individualized antiplatelet treatment.

Cardiovascular disease is prevalent and recurrent cardiovascular events are a major cause of morbidity and mortality. An expanding armamentarium of therapeutic options that reduce cardiovascular risk underscores the promise of precision medicine that effectively tailors therapy to subsequent risk. Clinical risk tools and currently available tests of platelet function are not sufficient to effectively guide therapy. The present invention features methods for identifying patients at high and low risk of subsequent cardiovascular events and to provide key preliminary data that will lead to subsequent trials designed to guide individualized treatment.

### Dual Antiplatelet Therapy Tradeoffs

Atherosclerosis is a systemic disease and coronary artery disease (CAD) is prevalent among patients with stroke. The high prevalence of non-calcified coronary plaque that is prone to rupture among patients with stroke caused by rupture of an intracranial artery suggests that plaques within the intracranial and coronary arteries behave in similar ways. The importance of plaque rupture as a cause of stroke in patients with intracranial atherosclerotic disease (ICAD) suggest that treatment strategies useful in the treatment of coronary artery disease (CAD) may be successful in patients with intracranial atherosclerotic disease (ICAD). In embodiments, subjects treated by any of the methods described herein have atherosclerosis in the central nervous system (e.g., intracranial atherosclerotic disease (ICAD)).

In the treatment of CAD, a strategy designed to reduce the risk of recurrent ischemic events is the continuation of dual antiplatelet therapy (DAPT) after heart attack caused by atherosclerotic plaque rupture. Long-term DAPT reduces the risk of subsequent cardiovascular events, however, this treatment is associated with an increased risk of bleeding. Accordingly, the benefits of long-term treatment with DAPT are at least partially offset by an increase in bleeding. Because of this trade-off, the use of DAPT long-term is limited in the care of patients with CAD. A strategy designed to limit the risk of bleeding events is the early transition (de-escalation) from DAPT to acetylsalicylic acid (ASA; aspirin) monotherapy. De-escalation is associated with a reduced incidence of bleeding, but also with increased incidence of thrombotic complications, such as stent thrombosis. In the treatment of patients with transient ischemic attack (TIA) and minor stroke, short-duration DAPT (≤1 month) started during the early acute ischemic phase is associated with less bleeding than longer DAPT and with a greater reduction of recurrent strokes compared with monotherapy.

### FcγRIIa

Not being bound by theory, FcγRIIa proteins on the surface of platelets can both activate platelets directly and amplify the activation of platelets. In embodiments, methods of the present invention include assessing patient risk for stroke or cardiovascular event based upon levels of the biomarker FcγRIIa protein. Using methods of the present invention, levels of FcγRIIa proteins can be used to identify patients at high and low risk of subsequent stroke or cardiovascular event. Unlike measures of platelet function that exhibit marked intra-individual variability, platelet surface levels of FcγRIIa proteins is a consistent marker of platelet reactivity.

Platelets from healthy young people can carry 1,000-4,000 molecules of FcγRIIa protein (Karas S P, Rosse W F, Kurlander R J. Characterization of the IgG-Fc receptor on human platelets. Blood 1982; 60:1277-82).

FcγRIIa protein levels can be measured using methods available in the art; for example, those methods described below and/or in U.S. Pat. No. 10,502,737 B2. FcγRIIa protein levels can be measured in a variety of biological samples including, as non-limiting examples, blood, serum, or plasma. Flow cytometry can be a powerful tool capable of assessing levels of FcγRIIa protein on platelet surfaces. Platelets can be identified by their size as well as a surface marker. External standards can enable the standardization of fluorescence intensity units in flow cytometry (e.g., Kay S, Herishanu Y, Pick M, Rogowski, O, Baron S, Naparstek E, Polliack A, Deutsch V R. Quantitative flow cytometry of ZAP-70 levels in chronic lymphocytic leukemia using molecules of equivalent soluble fluorochrome. Cytometry 2006; 70B:218-226; and Quadrini K J, Hegelund A C, Cortes K E, Xue C, Kennelly S M, Ji H, Högerkorp C-M, Mc Closky T W. Validation of a flow cytometry-based assay to assess C5aR receptor occupancy on neutrophils and monocytes for use in drug development. Cytometry B Clin Cytom 2016; 90B:177-190). Accordingly, platelet levels of FcγRIIa protein (e.g., the number of protein molecules on the surface of a platelet(s)) can be quantified with the use of external standards to quantify the number of molecules on the platelet surface rather than relative platelet levels of FcγRIIa. In various embodiments, the methods of the invention involve quantitating the number of FcγRIIa protein molecules on the surface of a platelet.

Not being bound by theory, because FcγRIIa protein amplifies platelet activation, increased platelet levels of FcγRIIa proteins can identify patients with consistently high platelet reactivity. Accordingly, high platelet levels of FcγRIIa leverages the clinical risk of increased platelet reactivity demonstrated by more than 100 studies and over 22,000 patients. Moreover quantification of platelet FcγRIIa levels eliminates factors that cause variability with platelet function test including the preparation of blood and the in vitro activation of platelets.

Not being bound by theory, FcγRIIa protein was identified as a low-affinity receptor for the fragment constant (Fc) portion of immunoglobulin (Ig) G. Platelets can coat an Ig-bound (opsonized) entity such as a bacterium via FcγRIIa proteins, and this binding triggers platelet activation and release of secondary mediators resulting in an amplification of the platelet response to a wide range of bacteria. In addition, platelet FcγRIIa protein is involved in heparin-induced thrombocytopenia and thrombosis. The principal cellular target for anti-platelet factor 4/heparin antibodies is the platelet FcγRIIa receptor protein. FcγRIIa protein and glycoprotein VI protein are linked on human platelets. Ligands acting at either receptor can activate dual proteolytic regulatory pathways. FcγRIIa protein can markedly enhance thrombus formation when platelets are perfused over a collagen-coated flow chamber under conditions of arterial and venous shear. Phosphorylation of FcγRIIa protein amplifies the activation of platelets. Platelets with more FcγRIIa proteins exhibit greater activation in response to sub-maximal concentrations of multiple agonists. FcγRIIa protein is a novel biomarker capable of identifying patients with increased platelet reactivity.

Platelet activation associated with a FcγRIIa protein can be reduced, inhibited, or ameliorated by anti-platelet agents and/or anticoagulants. Antiplatelet agents include, as non-limiting examples, those agents that reduce expression and/or signaling activity associated with protease-activated (PAR) proteins and/or adenosine diphosphate (ADP) receptor proteins. ADP receptor protein antagonists include, as non-limiting examples, the compounds prasugrel, clopidogrel, ticagrelor, ticlopidine, and other thienopyridines. PAR protein antagonists include, as a non-limiting example, vorapaxar (SCH 530348). Further non-limiting examples of anti-platelet agents include acetylsalicylic acid (ASA), dipyridamole, and eptifibatide. Anticoagulants include, as non-limiting examples, apixaban, argatroban, betrixaban, bivalirudin, dabigatran, desirudin, edoxaban, enoxaparin, heparin, reteplase, rivaroxaban, warfarin, and factor XIa (FXIa) inhibitors.

FcγRIIa Protein Levels and Stroke and/or Cardiovascular Event

Increased platelet reactivity can identify patients with minor stroke or TIA who are at greater risk of subsequent cerebrovascular event (e.g., stroke). Platelet testing can identify risk but cannot effectively guide selection of an antiplatelet therapy. Clinical trials have failed to demonstrate that currently available tests of platelet function can be used to guide treatment. Thus, currently available platelet function tests are not capable of guiding individualized care.

Not being bound by theory, FcγRIIa protein levels are likely a more precise indicator of risk for stroke than traditional platelet function tests. Platelet levels of FcγRIIa protein can be determined by megakaryocyte production, which is increased by interferon γ. The effect of FcγRIIa proteins on platelet function can be consistent throughout the life of a platelet. Not being bound by theory, FcγRIIa proteins can be stably displayed on the surface of a platelet. Average change in platelet levels of FcγRIIa proteins over a period of from about 6 to about 62 weeks can be less than about 10%.

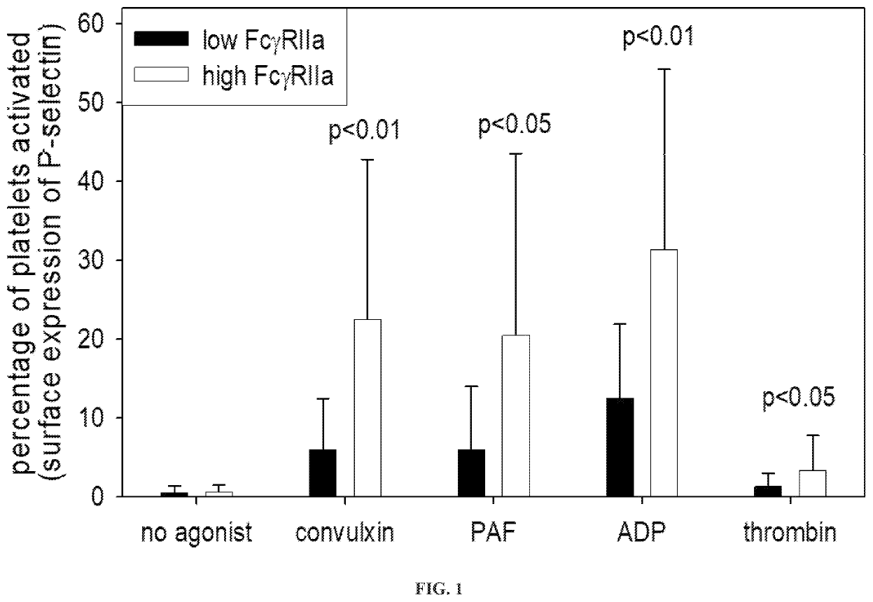

Unlike currently available tests of platelet function that measure platelet reactivity in response to a select agonist or group of agonists, platelet levels of FcγRIIa protein predicts increased platelet reactivity in response to a variety of agonists (FIG. 1).

Not being bound by theory, because determination of platelet FcγRIIa protein levels entails quantification of a surface marker, it is less sensitive to perturbations related to assay conditions (Table 2; see also J Thromb Thrombolysis 2019 July; 48(1):88-94). Neither anticoagulants nor a P2Y₁₂ antagonist alters platelet levels of FcγRIIa protein (Table 2).

In Table 1, blood was taken from 3 subjects and anticoagulated with corn trypsin inhibitor (32 μg/ml), trisodium citrate (3.2%), unfractionated heparin (1.2 U/ml), and bivalirudin (8 μg/ml). Cangrelor (500 nM) was added to blood anticoagulated with citrate. Duplicate determination of platelet FcγRIIa protein level was determined in each condition.

TABLE 2
Intra-assay coefficient of variation (CV)
for assay of platelet FcγRIIa protein level

| | |
|---|---|
| CV same anticoagulant | 1.9% ± 0.5% |
| CV between anticoagulants | 1.7% ± 0.5% |
| CV citrate anticoagulant vs citrate + cangrelor | 0.7% ± 0.2% |

### Diagnostics

The present invention features methods for the selection of subjects for treatment, where selected subjects have an increased level of FcγRIIa proteins on their platelets relative to a reference, which is indicative increased risk for stroke, cardiovascular event, and/or death. In embodiments, an increased level of FcγRIIa protein is indicative of increased risk for a subsequent stroke, cardiovascular event, and/or death following a previous stroke and/or cardiovascular event. Any suitable method can be used to detect platelet FcγRIIa protein in a subject sample and used to characterize subject risk for stroke, cardiovascular event, and/or death. Biological samples include bodily fluids (e.g., blood, blood serum, plasma, amniotic fluid, sputum, urine, cerebrospinal fluid, lymph, tear fluid, feces, saliva, or gastric fluid). Successful practice of the invention can be achieved with one or a combination of methods that can detect and/or quantify platelet FcγRIIa protein. Immunoassays in various formats (e.g., flow cytometry, ELISA) are non-limiting examples of methods for detection of analytes captured on a solid phase. Such methods can involve use of an FcγRIIa protein-specific antibody.

Virtually any method known in the art can be used to detect FcγRIIa proteins. For example, levels of platelet FcγRIIa proteins are compared by procedures well known in the art, such as flow cytometry, immunoassay, ELISA, western blotting, radioimmunoassay, immunocytochemistry, binding to magnetic and/or antibody-coated beads, in situ hybridization, fluorescence in situ hybridization (FISH), flow chamber adhesion assay, microarray analysis, or colorimetric assays. Methods may further include, one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ⁿ, quadrupole mass spectrometry, Fourier transform mass spectrometry (FTMS), and ion trap mass spectrometry, where n is an integer greater than zero. Detection methods may include use of a biochip array. Biochip arrays useful in the invention include protein and polynucleotide arrays. One or more markers are captured on the biochip array and subjected to analysis to detect the level of the markers in a sample.

Platelet FcγRIIa proteins may be captured with capture reagents fixed to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes are detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltammetry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et ah, Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein. The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). In one embodiment, the MS analysis is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

In other embodiments, levels of FcγRIIa proteins are detected in combination with one or more additional markers. While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, described herein provide for the measurement of more than one marker or clinical parameter.

The use of multiple markers increases the predictive value of the test and provides greater utility in diagnosis, toxicology, patient stratification and patient monitoring. The process called “Pattern recognition” detects the patterns formed by multiple markers. The inclusion of additional markers may improve the sensitivity and specificity in determining a patient's risk for developing a thrombotic disease or disorder associated with an undesirable increase in platelet reactivity. Subtle variations in data from clinical samples indicate that certain patterns of protein level or expression (e.g., FcγRIIa protein level) can predict phenotypes such as an increase in platelet reactivity, or can identify a patient that could benefit from more aggressive/powerful drug treatments (e.g., treatment with a more powerful anti-platelet agent, such as clopidogrel, dipyridamole, eptifibatide, prasugrel, ticagrelor, ticlopidine, or vorapaxar, and/or treatment with an anticoagulant, such as apixaban, argatroban, betrixaban, bivalirudin, dabigatran, desirudin, edoxaban, enoxaparin, heparin, reteplase, rivaroxaban, warfarin, and factor XIa (FXIa) inhibitors). Levels of platelet FcγRIIa proteins are correlated with platelet reactivity, and thus are useful in diagnosis. Antibodies that specifically bind FcγRIIa protein, or any other method known in the art may be used to monitor levels of platelet FcγRIIa protein. Detection of an alteration relative to a normal, reference sample can be used as a diagnostic indicator of platelet reactivity. In particular embodiments, a 2, 3, 4, 5, or 6-fold change in the level of platelet FcγRIIa protein is indicative of platelet reactivity and corresponds with an elevated level of platelet FcγRIIa protein.

In one embodiment, the level of platelet FcγRIIa protein is measured on at least two different occasions and an alteration in the levels as compared to normal reference levels over time is used as an indicator of risk for increased risk for stroke, cardiovascular event, and/or death. In embodiments, an alteration in levels (e.g., an increase) indicative of increased risk for a subsequent stroke, cardiovascular event, and/or death following a previous stroke and/or cardiovascular event. In general, levels of platelet FcγRIIa protein are present at low levels (about 6,000 copies per platelet) in a healthy subject (i.e., those who do not have reactive platelets). In one embodiment an increased level of platelet FcγRIIa protein is indicative of increased risk for stroke, cardiovascular event, and/or death. The increased/elevated level of platelet FcγRIIa protein can be about or at least about a threshold value of 7,000 molecules/platelet, 7,500 molecules/platelet, 8,000 molecules/platelet, 9,000 molecules/platelet, 10,000 molecules/platelet, 11,000 molecules/platelet, 12,000 molecules/platelet, 13,000 molecules/platelet, 14,000 molecules/platelet, or 15,000 molecules/platelet. Levels of FcγRIIa protein below a threshold value of about 7,000 molecules/platelet, 8,000 molecules/platelet, 9,000 molecules/platelet, 10,000 molecules/platelet, 11,000 molecules/platelet, 12,000 molecules/platelet, 13,000 molecules/platelet, 14,000 molecules/platelet, or 15,000 molecules/platelet can be indicative of a lower or low risk for stroke, cardiovascular event, and/or death. The increased level of platelet FcγRIIa protein can be about or at least about a 1.5-fold, 2-fold, 3-fold, 4-fold, 5-fold, 6-fold, 7-fold, 8-fold, 9-fold, or 10-fold increase relative to a reference. FcγRIIa protein can be measured using FACS analysis.

In embodiments, consistently elevated levels of platelet FcγRIIa protein are indicative of heightened risk for stroke, cardiovascular event, and/or death. In embodiments, levels of platelet FcγRIIa protein are considered as consistently elevated if the levels are above one or more of the above-listed threshold values at at least two measurement time points separated by about or at least about 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 17 weeks, 18 weeks, 19 weeks, 20 weeks, 21 weeks, 22 weeks, 23 weeks, 24 weeks, 25 weeks, 26 weeks, 7 months, 8 months, 9 months, 10 months, 11 months, or 12 months. In embodiments, lack of consistently elevated levels indicates a lower risk for stroke, cardiovascular event, or death relative to a subject with consistently elevated levels.

The diagnostic methods described herein can be used individually or in combination with any other diagnostic method described herein for a more accurate diagnosis of risk for stroke, cardiovascular event, and/or death.

A correlation between FcγRIIa protein levels and risk for stroke, cardiovascular event, and/or death can take into account the amount of platelet FcγRIIa protein in a sample compared to a control amount of platelet FcγRIIa protein (e.g., in normal subjects or in subjects where platelet reactivity is undetected). The correlation can be used to assess patient risk for stroke, cardiovascular event, and/or death, optionally where the event is a subsequent event following a previous stroke and/or cardiovascular event. A control can be, e.g., the average or median amount of platelet FcγRIIa protein present in comparable samples of normal subjects. The control amount is measured under the same or substantially similar experimental conditions as in measuring the test amount. As a result, the control can be employed as a reference standard, where the normal phenotype is known, and each result can be compared to that standard, rather than re-running a control.

Accordingly, a marker profile may be obtained from a subject sample and compared to a reference value obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of risk for stroke, cardiovascular event, and/or death.

In certain embodiments, the methods further comprise selecting antithrombotic therapy for administration to a subject with elevated levels of FcγRIIa protein on platelets. The treatment can be selected from, as non-limiting examples, anti-platelet agents, such as acetylsalicylic acid (ASA), clopidogrel, dipyridamole, eptifibatide, prasugrel, ticagrelor, ticlopidine, or vorapaxar, and/or anticoagulants such as apixaban, argatroban, betrixaban, bivalirudin, dabigatran, desirudin, edoxaban, enoxaparin, heparin, reteplase, rivaroxaban, warfarin, and factor XIa (FXIa) inhibitors. The invention also provides for such methods where platelet FcγRIIa protein is measured again after administration of a drug or therapy. In these cases, the methods are used to monitor patient status.

### Antibodies

As reported herein, antibodies that specifically bind FcγRIIa proteins are useful in diagnostic, as well as therapeutic methods. For example, antibodies that act as platelet FcγRIIa protein antagonists (e.g., IV.3 Fab) can be useful in the methods of the invention. In particular embodiments, the invention provides methods of using anti-platelet FcγRIIa protein antibodies for the inhibition of platelet reactivity. IV.3 is a monoclonal anti-FcγRIIa protein antibody that inhibits the phosphorylation of platelet FcγRIIa proteins during platelet activation. In embodiments, the invention provides antibodies and/or FcγRIIa-binding conjugates (e.g., an antibody conjugated to a detectable label) for use in detecting an FcγRIIa protein molecule.

In embodiments, the antibody is an antibody conjugate; e.g., an FcγRIIa-binding conjugate. In some instances, the FcγRIIa-binding antibody is conjugated to a detectable label (e.g., a fluorescent label).

Antibodies, or fragments thereof, useful for detection of an FcγRIIa protein molecule include commercially available antibodies. Non-limiting examples of commercially available antibodies, or fragments thereof, optionally conjugated to a detectable label, that bind FcγRIIa and can be used for detection of a FcγRIIa protein molecule include the FLI8.26 monoclonal antibody available from Fisher Scientific (Catalog number (Cat #) BDB550586) and Biosciences (Cat #550586); and CD32 polyclonal antibody available from Bioss (Cat #BS-2573R). Further non-limiting examples of antibodies, or fragments thereof, optionally conjugated to a detectable label, that bind FcγRIIa and can be used for detection of a FcγRIIa protein molecule include the following antibodies available from Invitrogen with the indicated catalog numbers: CD32 Monoclonal Antibody (6C4 (CD32)), Functional Grade, eBioscience™ (Cat #16-0329-81); CD32 Monoclonal Antibody (6C4 (CD32)), FITC, eBioscience™ (Cat #11-0329-42); CD32 Monoclonal Antibody (6C4 (CD32)), eFluor 450, eBioscience™ (Cat #48-0329-42); CD32 Monoclonal Antibody (6C4 (CD32)), APC, eBioscience™ (Cat #17-0329-42); CD32 Monoclonal Antibody (6C4 (CD32)), PE-Cyanine7, eBioscience™ (Cat #25-0329-42); CD32 Monoclonal Antibody (6C4 (CD32)), PE, eBioscience™ (Cat #12-0329-42); CD32 Monoclonal Antibody (6C4 (CD32)), PerCP-eFluor 710, eBioscience™ (Cat #46-0329-42); CD32 Monoclonal Antibody (AT10) (Cat #MA1-81191); CD32 Monoclonal Antibody (AT10) (Cat #MA1-81209); CD32 Recombinant Rabbit Monoclonal Antibody (JM10-70) (Cat #MA5-32601); CD32 Polyclonal Antibody (Cat #PA5-114980); CD32/FCGR2 Polyclonal Antibody (Cat #PA5-102032); CD32 Monoclonal Antibody (6C4 (CD32)), PE-Cyanine5.5, eBioscience™ (Cat #35-0329-42); CD32 Monoclonal Antibody (6C4 (CD32)), Biotin, eBioscience™ (Cat #13-0329-82); CD32 Monoclonal Antibody (CCG36), FITC (Cat #MA5-28346); CD32 Monoclonal Antibody (CCG36), PE (Cat #MA5-28347); CD32 Monoclonal Antibody (CCG39), FITC (Cat #MA5-28348); CD32 Monoclonal Antibody (CCG36) (Cat #MA5-28349); CD32 Monoclonal Antibody (CCG39) (Cat #MA5-28350); CD32-like Monoclonal Antibody (VPM63) (Cat #MA5-28351); Phospho-CD32 (Tyr288) Polyclonal Antibody (Cat #PA5-105099); CD32 Polyclonal Antibody (Cat #PA5-116206); CD32 Polyclonal Antibody (Cat #PA5-77978); and CD32 Polyclonal Antibody (Cat #PA5-87604). Other antibodies useful in the invention are those that attenuate platelet FcγRIIa protein signaling.

Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of an intact antibody, clear more rapidly from the circulation, and may have less non-specific tissue binding than an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion proteins, and unconventional antibodies.

Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5): 1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv protein antibodies include a covalently linked VH::VL heterodimer which can be expressed from a nucleic acid including VR- and VL-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds platelet FcγRIIa proteins is monoclonal. Alternatively, the anti-platelet FcγRIIa protein antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)2” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing soluble proteins, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding human FcγRIIa protein or immunogenic fragments thereof, can be provided to the host in a delivery vehicle that is taken up by immune cells of the host. The cells will in turn express the human FcγRIIa protein thereby generating an immunogenic response in the host. Alternatively, nucleic acid sequences encoding human FcγRIIa protein or immunogenic fragments thereof, can be expressed in cells in vitro, followed by isolation of the human FcγRIIa protein and administration of the FcγRIIa protein to a suitable host in which antibodies are raised.

Alternatively, antibodies against platelet FcγRIIa protein may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567; 5,530,101; 5,225,539; 5,585,089; 5,693,762; and 5,859,205.

### Inhibitory Nucleic Acids

Inhibitory nucleic acid molecules are those oligonucleotides that alter the levels or activity of platelet FcγRIIa protein for the treatment and/or prevention of stroke, cardiovascular event, death, and related disorders. Such oligonucleotides include single and double stranded nucleic acid molecules (e.g., DNA, RNA, and analogs thereof) that bind a nucleic acid molecule that encodes FcγRIIa (e.g., antisense molecules, siRNA, shRNA) as well as nucleic acid molecules that bind directly to a platelet FcγRIIa protein to modulate its biological activity (e.g., aptamers). Such inhibitory nucleic acid molecules reduce levels of FcγRIIa protein or polynucleotide in megakaryocytes and, accordingly, result in a reduction in FcγRIIa protein in platelets.

### Ribozymes

Catalytic RNA molecules or ribozymes that target an antisense FcγRIIa polynucleotide sequence of the present invention can be used to inhibit expression of a FcγRIIa polynucleotide in vivo. Such ribozymes reduce levels of FcγRIIa protein or polynucleotide in megakaryocytes and, accordingly, result in a reduction in FcγRIIa protein in platelets. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs contain a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in references cited herein and are familiar to one of skill in the art.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002). Given the sequence of a target gene, siRNAs may be designed to inactivate that gene (e.g., a gene encoding FcγRIIa). In embodiments, siRNAs reduce levels of FcγRIIa protein or polynucleotide in megakaryocytes and, accordingly, result in a reduction in FcγRIIa protein in platelets. Such siRNAs, for example, can be administered directly to an affected tissue, or administered systemically. The nucleic acid sequence of an Pari gene can be used to design small interfering RNAs (siRNAs). The 21 to 25 nucleotide siRNAs may be used, for example, as therapeutics to treat lupus.

The inhibitory nucleic acid molecules of the present invention may be employed as double-stranded RNAs for RNA interference (RNAi)-mediated knock-down of platelet FcγRIIa polynucleotide and/or protein expression. In one embodiment, platelet FcγRIIa protein and/or polynucleotide expression is reduced in megakaryocytes.

RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference in its entirety for all purposes).

### Test Samples

Methods and compositions of the invention are useful for the identification of an analyte (platelet FcγRIIa proteins) in a test sample. In one embodiment, the methods of the invention are suitable for detecting analytes of biological origin. Test samples include, but are not limited to, any liquid containing a dissolved or dispersed analyte (FcγRIIa proteins) of biological origin. Exemplary test samples include body fluids (e.g. blood, blood serum, plasma, amniotic fluid, sputum, urine, cerebrospinal fluid, lymph, tear fluid, feces, saliva, or gastric fluid), tissue extracts, or any liquid or biologic fluid containing a platelet. If the test sample is not in itself sufficiently fluid for the present purpose, it may be admixed with a suitable fluid to the desired fluidity, for instance by homogenization.

### Treatments

The methods described herein can be used for selecting, and then optionally administering, an optimal treatment for a subject. The subject (e.g., a patient having suffered a stroke or cardiovascular event) can be selected based upon having elevated levels of platelet FcγRIIa protein relative to a reference level. Thus the methods described herein include methods for the treatment and/or prevention of stroke, cardiovascular event, and/or death. Generally, the methods include administering a therapeutically effective amount of a treatment as described herein, to a subject who is in need thereof, or who has been determined to be in need of, such treatment. The treatments comprise administration of an anti-platelet agent, an anticoagulant agent, and/or any of the agents described herein. Non-limiting examples of anti-platelet agents include acetylsalicylic acid (ASA), clopidogrel, dipyridamole, eptifibatide, prasugrel, ticagrelor, ticlopidine, and vorapaxar. Non-limiting examples of anticoagulants include apixaban, argatroban, betrixaban, bivalirudin, dabigatran, desirudin, edoxaban, enoxaparin, heparin, reteplase, rivaroxaban, warfarin, and factor XIa (FXIa) inhibitors (see, e.g., those described in Rami A. Al-Horani and Umesh R. Desani, Expert Opin Ther Pat. 26:323-345 (2016)).

An effective amount of an agent can be administered in one or more administrations, applications or dosages. A therapeutically effective amount of a therapeutic compound (i.e., an effective dosage) depends on the therapeutic compounds selected. The compositions are administered from one or more times per day to one or more times per week; including once every other day. The skilled artisan will appreciate that certain factors may influence the dosage and timing to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions

Agents of the present disclosure are incorporated into a variety of formulations for therapeutic use (e.g., by administration) or in the manufacture of a medicament (e.g., for treating and/or preventing a stroke, cardiovascular event, and/or death or preventing a recurrence or subsequent occurrence thereof) by combining the agents with appropriate pharmaceutically acceptable carriers or diluents, and may be formulated into preparations in solid, semi-solid, liquid or gaseous forms. In some embodiments, the agent is an anti-platelet agent, such as acetylsalicylic acid (ASA), clopidogrel, dipyridamole, eptifibatide, prasugrel, ticagrelor, ticlopidine, or vorapaxar. In embodiments, the agent contains acetylsalicylic acid (ASA). In some embodiments the agent is an anticoagulant, such as apixaban, argatroban, betrixaban, bivalirudin, dabigatran, desirudin, edoxaban, enoxaparin, heparin, reteplase, rivaroxaban, warfarin, and factor XIa (FXIa) inhibitors (see, e.g., those described in Rami A. Al-Horani and Umesh R. Desani, Expert Opin Ther Pat. 26:323-345 (2016)). In some embodiments, the agent is ADP receptor antagonist (e.g., prasugrel, clopidogrel, ticagrelor, ticlopidine, and other thienopyridines), or a PAR antagonist (e.g., vorapaxar (SCH 530348)). Examples of formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration The diluent is selected so as not to affect the biological activity of the combination. Examples of such diluents include. without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents. toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents. stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade) Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under good manufacturing practice (GMP) conditions.

Formulations may be optimized for retention and stabilization in a subject and/or tissue of a subject, e.g., to prevent rapid clearance of a formulation by the subject. Stabilization techniques include cross-linking. multimerizing, or linking to groups such as polyethylene glycol. polyacrylamide, neutral protein carriers, etc. in order to achieve an increase in molecular weight.

Strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein (e.g., acetylsalicylic acid (ASA), clopidogrel, dipyridamole, eptifibatide, prasugrel, ticagrelor, ticlopidine, vorapaxar, apixaban, argatroban, betrixaban, bivalirudin, dabigatran, desirudin, edoxaban, enoxaparin, heparin, reteplase, rivaroxaban, warfarin, and factor XIa (FXIa) inhibitors) may be used (e.g., administered to an individual, such as a human individual) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes. The agent can be an ADP receptor antagonist or a PAR antagonist.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial high dose of an agent of the disclosure followed by a periodically administered maintenance dose. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein into association with a carrier or excipient, and/or one or more other accessory ingredients, and then, if desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methyl cellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it can be desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be advantageous.

Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration.

Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Agents provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent corresponding to an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent described herein.

As noted elsewhere herein, a drug of the instant disclosure may be administered via a number of routes of administration, including but not limited to subcutaneous, intravenous, intrathecal, intramuscular, intranasal, oral, transepidermal, parenteral, by inhalation, or intracerebroventricular.

In some embodiments of the present disclosure, a formulation as herein defined is administered to the subject by bolus administration.

Agents determined by a skilled clinician to be effective can be administered to the subject in an amount sufficient to achieve a desired effect at a desired site (e.g., reduction in occurrence, recurrence, or subsequent occurrence of stroke, cardiovascular event, and/or death). In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 ag/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 ag and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment can be sustained until a desired suppression of disease symptoms occurs. However, other dosage regimens may be useful. The progress of therapy can be monitored by conventional techniques and assays and/or by methods provided herein.

### Combination Treatments

It will be also appreciated that an agent or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity, potency, and/or efficacy in treating a disease in a subject in need thereof, in preventing a disease in a subject in need thereof, and/or in reducing the risk of developing a disease in a subject in need thereof. In certain embodiments, a pharmaceutical composition described herein including an agent described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of an agent of the disclosure. In some embodiments, the second therapeutic agent can be selected from one or more of an antioxidant, an anti-inflammatory agent, an antimicrobial, a steroid, etc.

In some embodiments, the agent or composition is administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic proteins or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to immunomodulatory agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the agents described herein or pharmaceutical compositions can be administered in combination with a medical procedure including, but not limited to, surgery.

### Hardware and Software

The present invention also relates to a computer system involved in carrying out the methods of the invention relating to subject and treatment selection. The computer system can be used, for example, to determine whether a subject has elevated levels and/or consistently elevated levels of FcγRIIa proteins. The computer system can use experimental data gathered by any of the methods described herein and determine from the data, according to any of the methods describe herein, the risk of a subject for stroke, cardiovascular event, and/or death.

A computer system (or digital device) may be used to receive, transmit, display and/or store results, analyze the results, and/or produce a report of results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

The computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

In another aspect, the invention provides kits for determining level of FcγRIIa protein on platelets in a sample and/or assessing risk of stroke, cardiovascular event, death, and the like. The kits can be used to detect biomarkers (e.g., FcγRIIa proteins) according to the invention. In one embodiment, the kit comprises agents that specifically recognize FcγRIIa proteins. In specific embodiments, the agents are antibodies. The kit can further include agents (e.g., any of those agents provided herein including, but not limited to, anti-platelet agents, such as acetylsalicylic acid (ASA), clopidogrel, dipyridamole, eptifibatide, prasugrel, ticagrelor, ticlopidine, or vorapaxar, and anti-coagulants such as apixaban, argatroban, betrixaban, bivalirudin, dabigatran, desirudin, edoxaban, enoxaparin, heparin, reteplase, rivaroxaban, warfarin, and factor XIa (FXIa) inhibitors) for administration to a subject determined to have elevated levels of FcγRIIa.

Fluorescently labeled antibody level are useful when flow cytometry methods are used to determine the level of FcγRIIa proteins on platelets in a sample. In a further embodiment, such a kit can comprise instructions for use in any of the methods described herein. In various embodiments, the instructions provide suitable operational parameters in the form of a label or separate insert. For example, the instructions may inform a consumer about how to collect the sample, how to wash the probe or the particular biomarkers to be detected, or how to determine platelet reactivity based on a measurement the level of FcγRIIa. In yet other embodiments, the kit can comprise one or more containers with controls (e.g., biomarker samples) to be used as standard(s) for calibration. In still other embodiments, the kit can comprise one or more therapeutic agents for the treatment of thrombosis (e.g., acetylsalicylic acid (ASA); ADP receptor antagonists such as prasugrel, clopidogrel, ticagrelor, ticlopidine, and other thienopyridines; PAR antagonists, such as vorapaxar; anticoagulants such as apixaban, argatroban, betrixaban, bivalirudin, dabigatran, desirudin, edoxaban, enoxaparin, heparin, reteplase, rivaroxaban, warfarin, and factor XIa (FXIa) inhibitors; and the like). In embodiments, the invention provides kits that include a test device for the detection of an analyte in a sample (see, e.g., U.S. Pat. No. 10,502,737B2). In one embodiment, the kit includes a lateral flow device described herein. In some embodiments, the kit comprises a container(s). Non-limiting examples of containers include boxes, ampoules, bottles, vials, tubes, bags, pouches, blister packs, or other suitable container forms known in the art. In one embodiment, such containers may be sterile. Such containers can be made of plastic, glass, laminated paper, metal foil, or other materials suitable for holding medicaments.

If desired the device is provided together with instructions for using it to identify the presence or absence of FcγRIIa protein in a sample and/or assess risk of stroke, cardiovascular event, and/or death, and the like. The instructions will generally include information about how to perform the methods described herein using components of the kit. The instructions may be printed directly on the container (when present), or as a label applied to the container, or as a separate sheet, pamphlet, card, or folder supplied in or with the container. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

If desired, the kit may also include a standard measure pipet, a test vial, and/or a liquid (e.g., ethanol, methanol, organic solvent, suitable buffer, such as phosphate buffered saline, or water) to be used in the extraction of a sample.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and proteins of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions and methods of the invention, and are not intended to limit the scope of what the inventors regard as their invention.

### EXAMPLES

### Example 1: Single Center Prospective Trial

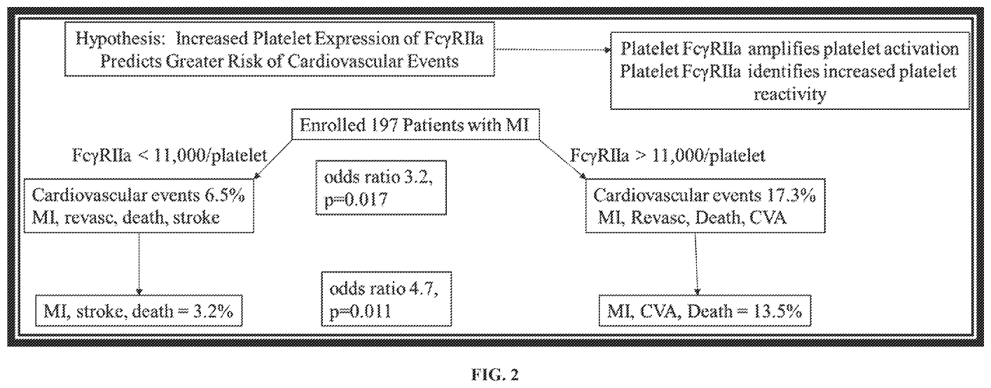

A single center prospective trial was completed to determine whether platelet levels of FcγRIIa proteins could identify patients with high and low cardiovascular risk. Patients (n=197) were enrolled shortly before discharge from a hospitalization for myocardial infarction (both ST elevation and non-ST elevation were included). All patients enrolled were treated with percutaneous coronary intervention (PCI). The primary endpoint (a composite of heart attack, stroke, death, and coronary revascularization) was lower in patients with platelet levels of FcγRIIa proteins <11,000 (FIG. 2). All patients were treated with acetylsalicylic acid (aspiring; ASA) (81 mg) and treatment with clopidogrel (˜64%) and ticagrelor (˜36%) was balanced in patients with high and low platelet levels of FcγRIIa proteins.

Clinical characteristics were well balanced with the exception of older age, diabetes, and prior revascularization being more prominent in the high levels group. Cox multivariate analysis for the combination of heart attack, stroke, revascularization, and death demonstrated a hazard ratio of 3.0 (p=0.02) for platelet levels of FcγRIIa proteins >11,000 when age, diabetes, and prior revascularization were included as covariates.

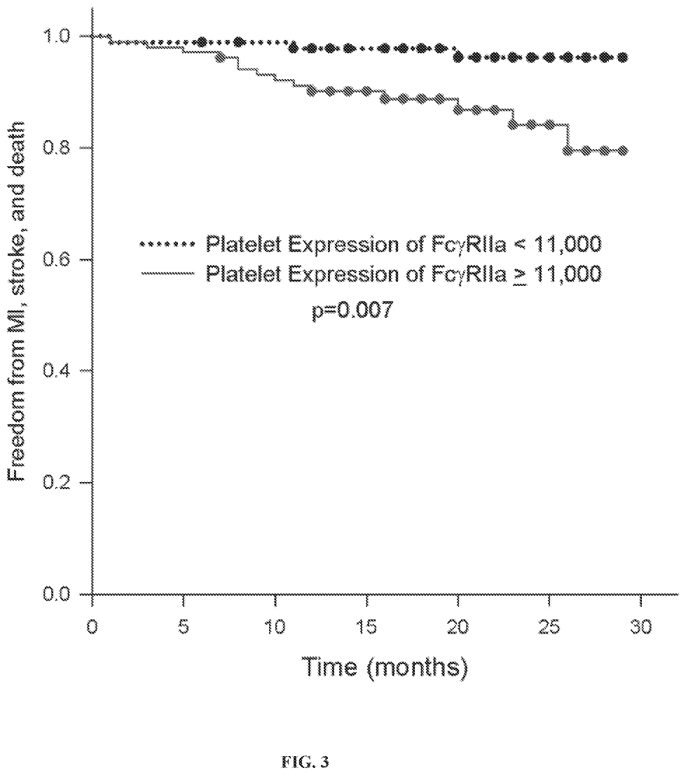

The incidence of revascularization was similar in patients with high and low platelet levels of FcγRIIa proteins (low platelet FcγRIIa proteins n=3, 3.2%; high platelet FcγRIIa proteins n=4, 3.8%, p=0.815). Patients with platelet levels of FcγRIIa proteins ≥11,000 had a greater risk of heart attack, stroke, and death that became apparent after 6 months (FIG. 3). Cox regression analysis was performed and platelet levels of FcγRIIa proteins was the sole covariate (hazard ratio 3.9, p=0.035) associated with freedom from myocardial infarction (MI), stroke, and death.

Predictive capacity of platelet levels of FcγRIIa proteins was evaluated. The sensitivity of high levels to identify patients with cardiovascular events was 0.82 (95% confidence intervals 0.57 to 0.92) and the specificity was 0.51 (95% confidence intervals 0.43 to 0.58). Cardiovascular events (heart attack, stroke, and death) were uncommon (8% of all patients experienced an event). The negative predictive value of low platelet levels of FcγRIIa proteins was 0.97 (95% confidence intervals 0.89 to 0.98) and the positive predictive value of high platelet levels of FcγRIIa proteins was 0.14 (95% confidence intervals 0.10 to 0.46).

A threshold of 11,000 molecules of FcγRIIa proteins/platelet was effective at identifying high and low risk of subsequent cardiovascular events in patients with heart attack. Platelet levels of FcγRIIa proteins can be considered a continuous variable. Not wishing to be bound by theory, the relationship between cardiovascular events and levels of FcγRIIa proteins may be continuous. In Table 3, FcγRIIa protein level is grouped to show the relationship between FcγRIIa protein level and risk of events. These results support the use of a threshold FcγRIIa protein level for assessment of patient risk.

TABLE 3
Association between platelet expression of
FcγRIIa protein and cardiovascular events

| | | Events | |
|---|---|---|---|
| FcγRIIa Protein | | (heart attack, | |
| Level | N | stroke, death) | rate |
| <8,000 | 18 | 0 | 0.0% |
| 8,000-10,000 | 48 | 3 | 6.3% |
| 10,000-12,000 | 66 | 8 | 12.1% |
| 12,000-14,000 | 42 | 4 | 9.5% |
| >14,000 | 23 | 2 | 8.7% |
| Total | 197 | 17 | 8.6% |

The results support a number of conclusions. Increased platelet FcγRIIa protein level was associated with consistently increased platelet reactivity. With calibration, platelet level of FcγRIIa protein was quantified to enable comparisons between samples and to assess changes over time. Unlike platelet function tests, measurement of platelet levels of FcγRIIa protein was not substantially affected by assay conditions. Platelet levels of FcγRIIa protein ≥11,000/platelet identified patients with an ˜4-fold greater risk of myocardial infarction (MI), stroke, and/or death.

These results may extend to patients with stroke caused by intracranial atherosclerotic disease (ICAD). These results support the use of FcγRIIa protein levels as a guide for individualized therapy.

### Example 2: Association of FcγRIIa Protein Level and Risk of Subsequent Stroke/Cardiovascular Events

A study is undertaken to compare the incidence of recurrent transient ischemic attack (TIA/stroke as well as myocardial infarction (MI) and death in patients whose platelet FcγRIIa protein level is above or below a threshold value of 11,000 FcγRIIa protein/platelet.

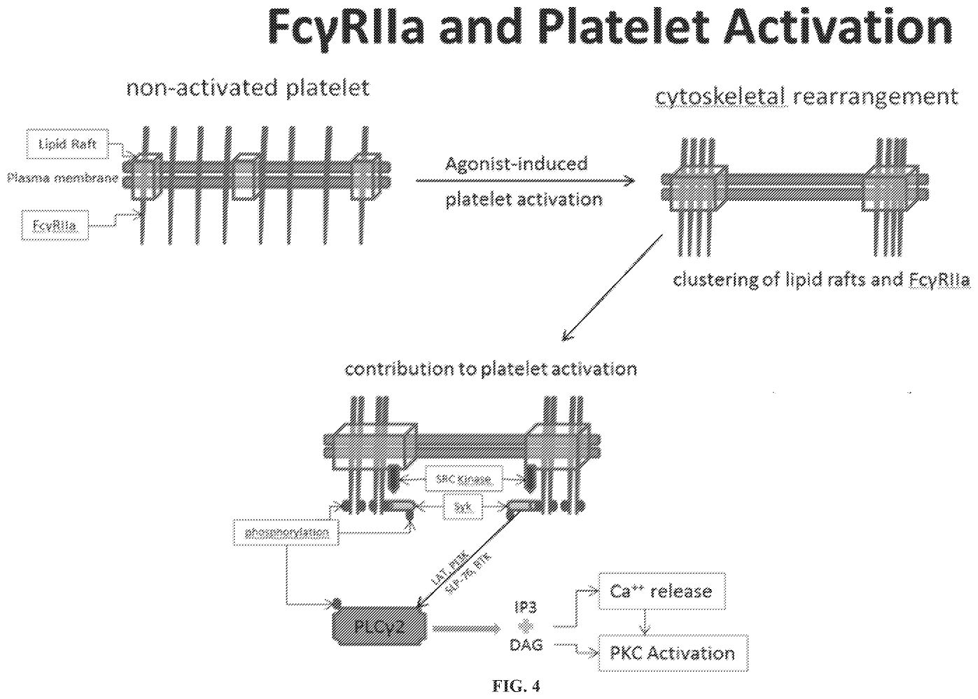

Flow cytometry is used to quantify platelet levels of FcγRIIa protein. Not being bound by theory, FcγRIIa protein is associated with platelet activation (FIG. 4). Blood is taken from patients with minor stroke/transient ischenic attack (TA) within 5 days of the primary event. Blood is taken a second time at the final visit. In each case, a 3 ml blue top vacutainer (trisodium citrate) is collected.

The assay described in Schneider D J, McMahon S R, Chava S, Taatjes-Sommer H S, Meagher S, Ehle G L, Brummel-Ziedins K E. FcγRIIa: A New Cardiovascular Risk Marker. J Am Coll Cardiol 2018; 72:237-238 is adapted to develop a lab assay involving the following steps: 1) Anticoagulation of blood with trisodium citrate (blue top vacutainer); 2) Addition of blood to a reaction tube with buffer and fluorochrome labelled anti-FcγRIIa protein antibody (Becton Dickinson Biosciences); 3) Fixation of platelets and lysis of red blood cells with Optilyse-C (Beckman Coulter); and 4) Analysis of samples with the use of flow cytometry after dilution with buffer. Kits are used to allow completion of steps 1-4. After step 3, analysis is performed up to 5 days later with a coefficient of variation of less than 10%. Flow cytometry is combined with standardization of output (Schneider D J, McMahon S R, Chava S, Taatjes-Sommer H S, Meagher S, Ehle G L, Brummel-Ziedins K E. FcγRIIa: A New Cardiovascular Risk Marker. J Am Coll Cardiol 2018; 72:237-238) to specifically quantify the number of molecules of FcγRIIa protein on the surface of platelets. Precise rather than qualitative assessment of FcγRIIa protein levels facilitates comparison between patients and over time. Non-limiting examples of strengths of the assay used to quantify platelet FcγRIIa protein levels can include 1) the standardization of output to enable specific quantification, 2) an intra-assay coefficient of variation less than 5% (McMahon S R, Chava S, Taatjes-Sommer H S, Meagher S, Brummel-Ziedins K E, Schneider D J. Variation in platelet levels of FcγRIIa protein after myocardial infarction. J Thromb Thrombolysis. 2019; 48:88-94), 3) the intra-individual coefficient of variation of ˜10% over the course of 1 month (McMahon, et al.) and 4) lack of effect of antiplatelet agents and anticoagulants on the assay results (McMahon, et al.).

Data analysis involves the following: 1) Hazard ratio of high vs low platelet FcγRIIa protein for primary endpoint; 2) Key covariates age, sex, diabetes mellitus, known atherosclerotic vascular disease (previous stroke, coronary artery disease (CAD), peripheral arterial disease); 3) Predictive analysis; 4) Assess both threshold and as continuous variable, include sensitivity analysis for threshold; 6) Compare levels at baseline to 1 year. The analysis takes into account a continuous or graded risk correlation with FcγRIIa protein level. A range will be identified over which patients have low risk or high risk for stroke, cardiovascular event, or death.

Platelet FcγRIIa protein level identifies patients at high and low risk of subsequent stroke/cardiovascular events. Low platelet FcγRIIa protein levels identifies patients at low risk of events, whereas high levels identifies patients at high risk of events. A low prevalence of stroke/cardiovascular events is observed among patients whose initial platelet FcγRIIa protein level was high but the second determination decreased to below a threshold for high levels (e.g., 11,000 FcγRIIa protein/platelet).

### Example 3: FcγRIIa Protein Level in Patients with Stroke

A study was undertaken to determine levels of FcγRIIa protein/platelet in 114 patients having had a stroke. Clinical characteristics of the patients are provided in Table 4 below. Platelet FcγRIIa exhibited a range of expression in patients with transient ischemic attack (TIA)/stroke of from 4,017 to 21,414 molecules/platelet (mean=11,092 SD=4017).

TABLE 4
Clinical characteristics of the 114 TIA/stroke patients

| | |
|---|---|
| Age | 68 ± 17 |
| Sex (% male) | 55% |
| Prior Stroke/TIA | 29% |
| Coronary artery disease (CAD) | 46% |
| Hypertensive heart disease (HTN) | 85% |
| Diabetes mellitus (DM) | 25% |

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The application may be related to U.S. Pat. No. 10,502,737 B2 and Liebeskind, et al., “Endothelial shear stress and platelet FcγRIIa expression in intracranial atherosclerotic disease,” Frontiers in Neurology, vol. 12, article 646309 (2021).

What is claimed is:

1. A method for treating a selected subject, the method comprising:
administering an antithrombotic agent to the selected subject who has previously had at least one stroke, wherein the subject is selected by determining that a level of FcγRIIa protein on platelets from the subject is increased relative to a reference, thereby treating the subject.

2. The method of claim 1, wherein the subject has intracranial atherosclerotic disease.

3. A method for treating a selected subject, the method comprising:
administering an antithrombotic agent to a subject who has intracranial atherosclerotic disease and has had at least one stroke, wherein the subject is selected by determining that a level of FcγRIIa protein on platelets from the subject is increased relative to a reference, thereby treating the subject.

4. The method of any one of claims 1-3, further comprising quantifying the number of molecules of FcγRIIa on individual platelets.

5. The method of any one of claims 1-4, wherein the stroke is a minor stroke and/or a transient ischemic attack.

6. The method of any one of claims 1-5, wherein the antithrombotic agent is selected from the group consisting of a small molecule compound, an inhibitory nucleic acid, and an antibody or antigen-binding fragment thereof.

7. The method of claim 6, wherein the inhibitory nucleic acid is selected from the group consisting of an antisense molecule, an shRNA, and an siRNA.

8. The method of any one of claims 1-7, wherein the antithrombotic agent comprises an antiplatelet agent or an anticoagulant.

9. The method of any one of claims 1-7, wherein the agent comprises an adenosine diphosphate (ADP) receptor antagonist and/or a protease-activated receptor (PAR) antagonist.

10. The method of any one of claims 1-9, wherein the anti-thrombotic agent comprises an ADP receptor antagonist.

11. The method of claim 10, wherein the ADP receptor antagonist targets P2Y₁₂.

12. The method of claim 10 or claim 11, wherein the ADP receptor antagonist comprises a small molecule compound.

13. The method of any one of claims 10-12, wherein the ADP receptor antagonist comprises a thienopyridine.

14. The method of claim 13, wherein the thienopyridine comprises prasugrel, clopidogrel, ticagrelor, or ticlopidine.

15. The method of any one of claims 1-14, wherein the antithrombotic agent comprises a PAR antagonist.

16. The method of claim 15, wherein the PAR antagonist targets PARI, PAR3, or PAR4.

17. The method of claim 15 or claim 16, wherein the PAR antagonist targets PARI.

18. The method of any one of claims 15-17, wherein the PAR antagonist comprises a small molecule compound.

19. The method of any one of claims 15-18, wherein the PAR antagonist comprises vorapaxar.

20. The method of any one of claims 1-19, wherein the antithrombotic agent comprises acetylsalicylic acid (ASA), dipyridamole, and/or eptifibatide.

21. The method of any one of claims 1-20, comprising administering at least two antithrombotic agents to the subject.

22. The method of any one of claims 1-8 or claim 21, wherein the antithrombotic agent comprises an anticoagulant agent.

23. The method of claim 22, wherein the anticoagulant is an inhibitor of factor XIa.

24. The method of claim 22, wherein the anticoagulant comprises apixaban, argatroban, betrixaban, bivalirudin, dabigatran, desirudin, edoxaban, enoxaparin, heparin, reteplase, rivaroxaban, and/or warfarin.

25. The method of any one of claims 1-24, wherein the level of FcyRIIa protein on platelets is determined using an assay selected from the group consisting of flow cytometry, immunoassay, ELISA, western blotting, and radioimmunoassay.

26. The method of any one of claims 1-25, wherein the level of FcyRIIa protein on platelets is determined using fluorometric or colorimetric assay.

27. The method of any one of claims 1-26, wherein determining the level of FcγRIIa protein on the platelets comprises contacting the platelets with a capture reagent.

28. The method of claim 21, wherein the capture reagent comprises an anti-FcγRIIa protein antibody or antigen-binding fragment thereof comprising a detectable label.

29. The method of claim 22, wherein the detectable label comprises a fluorochrome.

30. The method of any one of claims 1-23, wherein the level of FcγRIIa protein on the platelets is determined using flow cytometry.

31. The method of any one of claims 1-30, wherein the reference is a healthy subject that has not had a stroke.

32. The method of any one of claims 1-31, wherein the reference is a healthy subject that does not have intracranial atherosclerotic disease.

33. The method of any one of claims 1-32, wherein the increase is by at least about 1.5, 2, 3, 4, or 5-fold.

34. The method of any one of claims 1-33, wherein the level of FcγRIIa protein on platelets is increased relative to the reference if greater than about 7,500, 8,000, 9,000, or 10,000 FcyRIIa protein molecules per platelet.

35. The method of any one of claims 1-34, wherein the level of FcγRIIa protein on platelets is increased relative to the reference if greater than about 8,000 FcyRIIa protein molecules per platelet.

36. The method of claim 33, wherein the level of FcγRIIa protein on platelets is increased relative to the reference if greater than about 11,000 FcyRIIa protein molecules per platelet.

37. The method of any one of claims 1-36, wherein the subject is selected only if the level of FcγRIIa on platelets from the subject is determined to be equal to or greater than about 11,000 copies of FcγRIIa per platelet at two time points.

38. The method of claim 37, wherein the time points are separated by at least about one day.

39. The method of claim 37 or claim 38, wherein the time points are separated by at least about 7 days.

40. The method of any one of claims 1-39, wherein incidence and/or severity of a cardiovascular event is reduced.

41. The method of any one of claims 1-40, wherein incidence and/or severity of a subsequent stroke is reduced.

42. The method of any one of claims 1-41, wherein incidence of death is reduced.

43. A method for treating a selected subject who has intracranial atherosclerotic disease and has had at least one stroke, the method comprising:
administering an anti-platelet agent, and/or an anticoagulant to the selected subject, wherein the subject is selected by determining a level of FcγRIIa on platelets from the subject, wherein a level greater than about 7,500 copies of FcγRIIa per platelet identifies the subject as at risk for subsequent stroke and/or cardiovascular event and in need of antithrombotic therapy.

44. The method of claim 43, further comprising quantifying the number of molecules of FcγRIIa protein on individual platelets.

45. The method of claim 43 or claim 44, wherein the anti-platelet agent comprises an Adenosine diphosphate (ADP) receptor antagonist, and/or a Protease-activated receptor (PAR) antagonist.

46. The method of claim 45, wherein the Adenosine diphosphate (ADP) receptor antagonist and/or the Protease-activated receptor (PAR) antagonist comprises one or more of prasugrel, ticagrelor, clopidogrel, and vorapaxar.

47. The method of any one of claims 43-46, wherein the anti-platelet agent comprises acetylsalicylic acid (ASA), dipyridamole, or eptifibatide.

48. The method of any one of claims 43-47, wherein the anticoagulant comprises one or more of apixaban, argatroban, betrixaban, bivalirudin, dabigatran, desirudin, edoxaban, enoxaparin, heparin, reteplase, rivaroxaban, and warfarin.

49. The method of any one of claims 43-48, wherein the level of the FcγRIIa is determined by contacting a sample comprising platelets from the subject with an FcγRIIa-binding conjugate to form a bound complex of the FcγRIIa-binding conjugate and an FcγRIIa protein molecule on the surface of the platelets, and detecting binding between the FcγRIIa-binding conjugate and the FcγRIIa protein molecule.

50. The method of claim 49, wherein the FcγRIIa-binding conjugate is an anti-FcγRIIa antibody.

51. The method of any one of claims 43-50, wherein the level of platelet FcγRIIa is determined using an assay selected from the group consisting of flow cytometry, immunoassay, ELISA, western blotting, and radioimmunoassay.

52. A kit for use in the method of any one of claims 1-51, wherein the kit comprises a FcγRIIa protein capture reagent.

53. The kit of claim 52, wherein the capture reagent comprises a fluorochrome-labeled antibody.


SEQUENCE LISTING (4 sequences)

SEQ ID NO: 1 - protein, 65 aa, Artificial Sequence
  Synthetic
VVYTDCTESGQNLCLCEGSNVCGQGNKCILGSDGEKNQCVTGEGTPKPQSHNDGDFEEIPEEYLQ

SEQ ID NO: 2 - protein, 355 aa, Artificial Sequence
  Synthetic
SYQGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 3 - protein, 317 aa, Homo sapiens
  SITE (1)..(317): FcyRIIa protein
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 4 - DNA, 2429 bp, Homo sapiens
  misc_feature (1)..(2429): FcyRIIa polynucleotide
ctcttttctaagcttgtctcttaaaacccactggacgttggcacagtgctgggatgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaaagagtaacgttatgccatgtggtcatactctcagcttgctgagtggatgacaaaaagaggggaattgttaaaggaaaatttaaatggagactggaaaaatcctgagcaaacaaaaccacctggcccttagaaatagctttaactttgcttaaactacaaacacaagcaaaacttcacggggtcatactacatacaagcataagcaaaacttaacttggatcatttctggtaaatgcttatgttagaaataagacaaccccagccaatcacaagcagcctactaacatataattaggtgactagggactttctaagaagatacctacccccaaaaaacaattatgtaattgaaaaccaaccgattgcctttattttgcttccacattttcccaataaatacttgcctgtgacattttgccactggaacactaaacttcatgaattgcgcctcagatttttcctttaacatcttttttttttttgacagagtctcaatctgttacccaggctggagtgcagtggtgctatcttggctcactgcaaacccgcctcccaggtttaagcgattctcatgcctcagcctcccagtagctgggattagaggcatgtgccatcatacccagctaatttttgtattttttattttttttttttagtagagacagggtttcgcaatgttggccaggccgatctcgaacttctggcctctagcgatctgcccgcctcggcctcccaaagtgctgggatgaccagcatcagccccaatgtccagcctctttaacatcttctttcctatgccctctctgtggatccctactgctggtttctgccttctccatgctgagaacaaaatcacctattcactgcttatgcagtcggaagctccagaagaacaaagagcccaattaccagaaccacattaagtctccattgttttgccttgggatttgagaagagaattagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgagaaacgcttatgttacaggttacatgagagcaatcatgtaagtctatatgacttcagaaatgttaaaatagactaacctctaacaacaaattaaaagtgattgtttcaaggtgatgcaattattgatgacctattttatttttctataatgatcatatattacctttgtaataaaacattataaccaaaaca